(19) United States
(12) Patent Application Publication
(10) Pub. No.: US 2009/0054489 A1
(43) Pub. Date: Feb. 26, 2009

(54) Multimediator Dopamine Transport Inhibitors, and Uses Related Thereto

(75) Inventor: James R. Hauske, La Jolla, CA (US)

Correspondence Address: ROPES & GRAY LLP, PATENT DOCKETING 39/41, ONE INTERNATIONAL PLACE, BOSTON, MA, 02110-2624, US

(73) Assignee: Prexa Pharmaceuticals, Inc., Boston, MA (US)

(21) Appl. No.: 11/884,961

(22) PCT Filed: Feb. 21, 2006
(86) PCT No.: PCT/US2006/006347

§ 371 Date: Jul. 28, 2008

Related U.S. Application Data
(60) Provisional application No. 60/656,021, filed on Feb. 23, 2005.

(51) Int. Cl. C07D 401/12; A61K 31/454; A61P 25/00; A61P 25/18; A61P 25/22; A61P 25/24; A61P 25/30; G06Q 90/00

(52) U.S. Cl. 514/326; 546/210; 546/208; 705/500

(57) ABSTRACT

The invention provides a class of DAT-5HT2 antagonists, packaged pharmaceuticals comprising such antagonists, and their uses in treating, or manufacturing medicaments for treating disease conditions, including a movement disorder, attention deficit disorder or attention-deficit hyperactivity disorder, anxiety, depression or psychotic disorder. Related business methods such as marketing the inhibitors to healthcare providers are also provided.

### BACKGROUND OF THE INVENTION

Major depression is characterized by feelings of intense sadness and despair, mental slowing and loss of concentration, pessimistic worry, agitation, and self-deprecation. Physical changes also occur, especially in severe or “melancholic” depression. These include insomnia or hypersomnia, anorexia and weight loss (or sometimes overeating), decreased energy and libido, and disruption of normal circadian rhythms of activity, body temperature, and many endocrine functions.

Treatment regimens commonly include the use of tricyclic antidepressants, monoamine oxidase inhibitors, some psychotropic drugs, lithium, and electroconvulsive therapy (ECT) (see R. J. Baldessarini in Goodman & Gilman's The Pharmacological Basis of Therapeutics, 9th Edition, Chapter 19, McGraw-Hill, 1996 for a review). More recently, new classes of antidepressant drugs are being developed including selective serotonin reuptake inhibitors (SSRIs), Specific monoamine reuptake inhibitors and 5-HT_{1A} receptor agonists, antagonists and partial agonists.

Anxiety is an emotional condition characterized by feelings such as apprehension and fear accompanied by physical symptoms such as tachycardia, increased respiration, sweating and tremor. It is a normal emotion but when it is severe and disabling it becomes pathological.

Anxiety disorders are generally treated using benzodiazepine sedative-antianxiety agents. Potent benzodiazepines are effective in panic disorder as well as in generalized anxiety disorder, however, the risks associated with drug dependency may limit their long-term use. 5-HT_{1A} receptor partial agonists also have useful anxiolytic and other psychotropic activity, and less likelihood of sedation and dependence (see R. J. Baldessarini in Goodman & Gilman's The Pharmacological Basis of Therapeutics, 9th Edition, Chapter 18, McGraw-Hill, 1996 for a review).

Bipolar Disorder is a psychiatric condition which is prevelant across cultures and age groups. The lifetime prevalence of Bipolar Disorder can be as high as 1.6%. DSM-IV, p. 353 (American Psychiatric Association, Washington, D.C. 1997). Bipolar Disorder is a recurrent disorder characterized by one or more Manic Episodes immediately before or after a Major Depressive Episode or may be characterized by one or more Major Depressive Episodes accompanied by at least one Hypomanic Episode. Additionally, the symptoms must cause clinically significant distress or impairment in social, occupational, or other important areas of functioning.

In some cases the Hypomanic Episodes themselves do not cause impairment; however, the impairment may result from the Major Depressive Episodes or from a chronic pattern of unpredictable mood episodes and fluctuating unreliable interpersonal and occupational functioning. The symptoms of Bipolar Disorder must not be better accounted for by a psychotic condition or due to the direct physiological effects of a medication, other somatic treatments for depression, drugs of abuse, or toxin exposure.

Bipolar Disorder is associated with a significant risk of completed suicide. Further, the patient suffering from Bipolar Disorder is likely to suffer from school truancy, school failure, occupational failure, or divorce.

Therefore, Bipolar Disorder is a serious, fairly prevelant, psychological condition which is clearly distinguished from psychotic conditions such as schizophrenia. DSM-IV, p. 353 (American Psychiatric Association, Washington, D.C. 1994). DSM-IV, p. 353 (American Psychiatric Association, Washington, D.C. 1994).

There remains a long felt need for treatments which provide a favorable safety profile and effectively provide relief for the patient suffering an anxiety, depression or psychotic condition.

### SUMMARY OF THE INVENTION

The present invention relates to compounds, packaged pharmaceuticals, and methods for treating patients suffering from an anxiety, depression or psychotic disorder. In particular, the invention relates to compounds having dopamine transport (DAT) inhibitory activity as well as 5HT₂ₐ receptor antagonist activity and/or 5HT_{2c} receptor antagonist activity. The invention also relates to uses of the compounds in the manufacture of pharmaceutical compositions, methods for conducting a pharmaceutical business, and methods for conducting a medical assistance reimbursement program.

The DAT-5HT2 antagonists of the present invention are represented by Formula I, or a pharmaceutically acceptable salt, solvate, metabolite or pro-drug thereof:

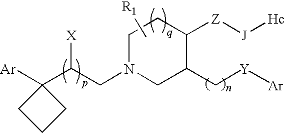

wherein, as valence and stability permit,
Ar, independently for each occurrence, represents a substituted or unsubstituted aryl or heteroaryl ring;
Hc represents a substituted or unsubstituted nitrogen-containing heteroaryl ring;
X represents H or OR;
Y and Z independently represent —O—, —S—, —C(—R)₂—, or —N(—R)—;
R, independently for each occurrence, represents H or lower alkyl;
R₁ represents one or more substituents, each independently selected from halogen, amino, acylamino, amidino, cyano, nitro, azido, ether, thioether, sulfoxido, -J-R₈, -J-OH, -J-lower alkyl, -J-lower alkenyl, -J-SH, -J-NH₂, or substituted or unsubstituted lower alkyl, lower alkenyl, cycloalkyl, heterocyclyl, cycloalkylalkyl, heterocyclylalkyl, aryl, heteroaryl, aralkyl, or heteroaralkyl, or protected forms of the above;
R₈, independently for each occurrence, represents H or substituted or unsubstituted lower alkyl, cycloalkyl, heterocyclyl, aralkyl, heteroaralkyl, aryl, or heteroaryl;
J represents, independently for each occurrence, a chain having 0-8 units selected from —C(—R)₂—, —N(—R)—, —O—, and —S—;
m is an integer from 0 to 2;
n is an integer from 0 to 2;
p is 0 or 1; and
q is an integer from 0 to 2, preferably 1,
and -Z-J-Hc, taken together, represent a substituted or unsubstituted nitrogen-containing heterocyclic or heteroaryl ring.

In another embodiment, the invention provides a packaged pharmaceutical comprising the DAT-5HT2 antagonist of the invention in an amount sufficient to treat an anxiety, depression or psychotic disorder and formulated in a pharmaceutically acceptable carrier; and instructions (written and/or pictorial) describing the use of the formulation for treating a patient. The packaged pharmaceutical may be provided in a once-a-day formulation. The packaged pharmaceutical may be formulated for oral administration or formulated as a transdermal patch. The packaged pharmaceutical may be provided in an escalating dose which produces an escalating serum concentration of said DAT-5HT2 antagonist(s) over a period of at least 4 hours.

In another embodiment, the invention provides a packaged pharmaceutical comprising (i) a mood-stabilizing formulation of a DAT-5HT2 antagonist of the invention, (ii) a second drug selected from the group consisting of a serotonin reuptake inhibitor, a 5HT₆ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, and a sedative-hypnotic drug, and (iii) a label indicating the use of the packaged pharmaceutical for use in the treatment of a patient suffering from an anxiety, depression or psychotic disorder. In some embodiments, the DAT-5HT2 antagonist formulation and the second drug are comingled in single dosage form.

In another embodiment, the invention provides for the use of a DAT-5HT2 antagonist of the invention in the manufacture of a pharmaceutical composition for prophylaxis or treatment of a patient susceptible to or suffering from a movement disorder.

In yet another embodiment, the invention provides a method for treating an anxiety, depression or psychotic disorder comprising administering to the patient a composition of a DAT-5HT2 antagonist of the invention. The method may be for the treatment of patients diagnosed with depression. The method may be for the treatment of depression selected from episodic or recurrent major depressive disorders, dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, seasonal affective disorder, or bipolar disorders or manic depression.

The method may be for the treatment of patients diagnosed with Bipolar Disorder, Bipolar Depression or Unipolar Depression or for the treatment of patients diagnosed with an anxiety disorder. The anxiety disorder may be selected from a obsessive-compulsive disorder, a panic disorder, a psychoactive substance anxiety disorder, a post-traumatic stress disorder, a generalized anxiety disorder, a anxiety disorder NOS, an organic anxiety disorder, a phobia, or a substance-induced anxiety. The substance-induced anxiety may be selected from alcohol, amphetamines, caffeine, cannabis, cocaine, hallucinogens, inhalants, phencycedine, sedatives, hypnotics, anxiolytics or other substance-induced, and adjustment disorders with anxiety or with mixed anxiety and depression.

The method may also be for the treatment of patients diagnosed with a psychotic disorder, and the psychotic disorder may be selected from schizophrenia, schizophreniform diseases, acute mania, schizoaffective disorders, and depression with psychotic features.

In another embodiment, the invention provides a packaged pharmaceutical comprising: a DAT-5HT2 antagonist of the invention in an amount sufficient to treat attention deficit disorder or attention-deficit hyperactivity disorder and formulated in a pharmaceutically acceptable carrier; and instructions (written and/or pictorial) describing the use of the formulation for treating a patient.

In another embodiment, the invention provides for the use of a DAT-5HT2 antagonist of the invention in the manufacture of a pharmaceutical composition for prophylaxis or treatment of a patient susceptible to or suffering from attention deficit disorder or attention-deficit hyperactivity disorder.

In another embodiment, the invention provides a method for treating attention deficit disorder or attention-deficit hyperactivity disorder comprising administering to the patient a composition of a DAT-5HT2 antagonist of the invention.

In another embodiment, the invention provides a method for conducting a pharmaceutical business, comprising: (a) manufacturing the packaged pharmaceutical of the invention; and (b) marketing to healthcare providers the benefits of using the package or preparation to treat patients suffering from an anxiety, depression or psychotic disorder, or from attention deficit disorder or attention-deficit hyperactivity disorder.

In another embodiment, the invention provides a method for conducting a pharmaceutical business, comprising: (a) providing a distribution network for selling the packaged pharmaceutical of the invention; and (b) providing instruction material to patients or physicians for using the package or preparation to treat patients suffering from an anxiety, depression or psychotic disorder, or from attention deficit disorder or attention-deficit hyperactivity disorder.

In another embodiment, the invention provides a method for conducting a pharmaceutical business, comprising: (a) determining an appropriate dosage of an DAT-5HT2 antagonist of the invention to enhance function performance in a class of patients suffering from an anxiety, depression or psychotic disorder, or from attention deficit disorder or attention-deficit hyperactivity disorder; (b) conducting therapeutic profiling of one or more formulations of the DAT-5HT2 antagonist identified in step (a), for efficacy and toxicity in animals; and (c) providing a distribution network for selling a the formulations identified in step (b) as having an acceptable therapeutic profile. The method may include an additional step of providing a sales group for marketing the preparation to healthcare providers.

In another embodiment, the invention provides a method for conducting a medical assistance reimbursement program, comprising: (a) providing a reimbursement program which permits, for prescription of a DAT-5HT2 antagonists of the invention for treating an anxiety, depression or psychotic disorder, or from attention deficit disorder or attention-deficit hyperactivity disorder, at least partial reimbursement to a healthcare provider or patient, or payment to a drug distributor; (b) processing one or more claims for prescription of an DAT-5HT2 antagonists for treating an anxiety, depression or psychotic disorder, or from attention deficit disorder or attention-deficit hyperactivity disorder; and (c) reimbursing the healthcare provider or patient, or paying a drug distributor, at least a portion of the cost of said prescription.

### BRIEF DESCRIPTION OF THE DRAWINGS

FIG. 1 shows two illustrative DAT-5HT2 antagonists, CNS-30,100 and CNS-31,100.

FIG. 2 shows in vitro profiles of two illustrative DAT-5HT2 antagonists, CNS-30,100 and CNS-31,100.

### DETAILED DESCRIPTION OF THE INVENTION

### I. Overview

The present invention relates to novel compounds for treating anxiety, depression or psychotic conditions. These diseases or disorders include, but are not limited to, single episodic or recurrent major depressive disorders, dysthymic disorders, depressive neurosis, neurotic depression, melancholic depression, atypical depression, anxiety and phobias, seasonal affective disorder, bipolar disorders, manic depression, unipolar depression, schizophrenia, schizophreniform diseases, acute mania, schizoaffective disorders, and depression with psychotic features.

The methods and formulations of the present invention can also be used to treat attention-deficit hyperactivity disorder.

### II. Definitions

The term “administering” means prescribing or providing medication in a dosage form and amount.

As used herein, the term “depression” includes depressive disorders, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, seasonal affective disorder, or bipolar disorders or manic depression, for example, bipolar I disorder, bipolar II disorder and cyclothymic disorder.

Other mood disorders encompassed within the term “depression” include dysthymic disorder with early or late onset and with or without atypical features; dementia of the Alzheimer's type, with early or late onset, with depressed mood; vascular dementia with depressed mood, disorders induced by alcohol, amphetamines, cocaine, hallucinogens, inhalants, opioids, phencyclidine, sedatives, hypnotics, anxiolytics and other substances; schizoaffective disorder of the depressed type; and adjustment disorder with depressed mood.

By “unipolar depression” or “major depressive disorder” is meant a clinical course that is characterized by one or more major depressive episodes in an individual without a history of manic, mixed, or hypomanic episodes. The diagnosis of unipolar depression is not made if: manic, mixed, or hypomanic episodes develop during the course of depression; if the depression is due to the direct physiological effects of a substance; if the depression is due to the direct physiological effects of a general medical condition; if the depression is due to a bereavement or other significant loss (“reactive depression”); or if the episodes are better accounted for by schizoaffective disorder and are not superimposed on schizophrenia, schizophreniform disorder, delusional disorder, or psychotic disorder. If manic, mixed, or hypomanic episodes develop, then the diagnosis is changed to a bipolar disorder. Depression may be associated with chronic general medical conditions (e.g., diabetes, myocardial infarction, carcinoma, stroke). Generally, unipolar depression is more severe than dysthymia.

The term “anxiety disorders” includes, but is not limited to obsessive-compulsive disorder, psychoactive substance anxiety disorder, post-traumatic stress disorder, generalized anxiety disorder, anxiety disorder NOS, and organic anxiety disorder. Anxiety disorders include panic disorder with or without agoraphobia, agoraphobia without history of panic disorder, specific phobias, for example, specific animal phobias, social phobias, obsessive-compulsive disorder, stress disorders including post-traumatic stress disorder and acute stress disorder, and generalized anxiety disorders. “Generalized anxiety” is typically defined as an extended period (e.g. at least six months) of excessive anxiety or worry with symptoms on most days of that period. The anxiety and worry is difficult to control and may be accompanied by restlessness, being easily fatigued, difficulty concentrating, irritability, muscle tension, and disturbed sleep. “Panic disorder” is defined as the presence of recurrent panic attacks followed by at least one month of persistent concern about having another panic attack. A “panic attack” is a discrete period in which there is a sudden onset of intense apprehension, fearfulness or terror. During a panic attack, the individual may experience a variety of symptoms including palpitations, sweating, trembling, shortness of breath, chest pain, nausea and dizziness. Panic disorder may occur with or without agoraphobia.

“Phobias” includes agoraphobia, specific phobias and social phobias. “Agoraphobia” is characterized by an anxiety about being in places or situations from which escape might be difficult or embarrassing or in which help may not be available in the event of a panic attack. Agoraphobia may occur without history of a panic attack. A “specific phobia” is characterized by clinically significant anxiety provoked by feared object or situation. Specific phobias include the following subtypes: animal type, cued by animals or insects; natural environment type, cued by objects in the natural environment, for example storms, heights or water; blood-injection-injury type, cued by the sight of blood or an injury or by seeing or receiving an injection or other invasive medical procedure; situational type, cued by a specific situation such as public transportation, tunnels, bridges, elevators, flying, driving or enclosed spaces; and other type where fear is cued by other stimuli. Specific phobias may also be referred to as simple phobias. A “social phobia” is characterized by clinically significant anxiety provoked by exposure to certain types of social or performance circumstances. Social phobia may also be referred to as social anxiety disorder.

Other anxiety disorders encompassed within the term “anxiety” include anxiety disorders induced by alcohol, amphetamines, caffeine, cannabis, cocaine, hallucinogens, inhalants, phencycedine, sedatives, hypnotics, anxiolytics and other substances, and adjustment disorders with anxiety or with mixed anxiety and depression.

Anxiety may be present with or without other disorders such as depression in mixed anxiety and depressive disorders. The compositions of the present invention are therefore useful in the treatment of anxiety with or without accompanying depression.

The term “psychotic disorder” includes, for example, schizophrenia, schizophreniform diseases, acute mania, schizoaffective disorders, and depression with psychotic features. The titles given these conditions represent multiple disease states. The following list illustrates a number of these disease states, many of which are classified in the Diagnostic and Statistical Manual of Mental Disorders, 4th Edition, published by the American Psychiatric Association (DSM). The DSM code numbers for these disease states are supplied below, when available, for the convenience of the reader: Paranoid Type Schizophrenia 295.30; Disorganized Type Schizophrenia 295.10; Catatonic Type Schizophrenia 295.20; Undifferentiated Type Schizophrenia 295.90; Residual Type Schizophrenia 295.60; Schizophreniform Disorder 295.40; Schizoaffective Disorder 295.70; Schizoaffective Disorder of the Depressive Type; and Major Depressive Disorder with Psychotic Features 296.24, 296.34.

By “attention-deficit hyperactivity disorder” or “ADHD” is meant a behavioral disorder characterized by a persistent and frequent pattern of developmentally inappropriate inattention, impulsivity, and hyperactivity. Indications of ADHD include lack of motor coordination, perceptual-motor dysfunctions, EEG abnormalities, emotional lability, opposition, anxiety, aggressiveness, low frustration tolerance, poor social skills and peer relationships, sleep disturbances, dysphoria, and mood swings (“Attention Deficit Disorder,” The Merck Manual of Diagnosis and Therapy (17th Ed.), eds. M. H. Beers and R. Berlow, Eds., 1999, Whitehouse Station, N.J.).

By “treating” is meant the medical management of a patient with the intent that a cure, amelioration, or prevention of a disease, pathological condition, or disorder will result. This term includes active treatment, that is, treatment directed specifically toward improvement of a disease, pathological condition, or disorder, and also includes causal treatment, that is, treatment directed toward removal of the cause of the disease, pathological condition, or disorder. In addition, this term includes palliative treatment, that is, treatment designed for the relief of symptoms rather than the curing of the disease, pathological condition, or disorder; preventive treatment, that is, treatment directed to prevention of the disease, pathological condition, or disorder; and supportive treatment, that is, treatment employed to supplement another specific therapy directed toward the improvement of the disease, pathological condition, or disorder. The term “treating” also includes symptomatic treatment, that is, treatment directed toward constitutional symptoms of the disease, pathological condition, or disorder.

The term “agonist” refers to a compound that mimics the action of natural transmitter or, when the natural transmitter is not known, causes changes at the receptor complex in the absence of other receptor ligands.

The term “antagonist” refers to a compound that binds to a receptor site, but does not cause any physiological changes unless another receptor ligand is present.

The term “ligand” refers to a compound that binds at the receptor site.

### III. Exemplary Formulations

The subject DAT-5HT2 agents are represented by Formula I, or are a pharmaceutically acceptable salt, solvate, metabolite or pro-drug thereof:

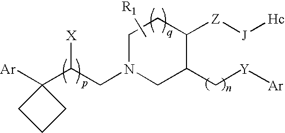

wherein, as valence and stability permit,

Ar, independently for each occurrence, represents a substituted or unsubstituted aryl or heteroaryl ring;

Hc represents a substituted or unsubstituted nitrogen-containing heteroaryl ring;

X represents H or OR;

Y and Z independently represent —O—, —S—, —C(—R)₂—, or —N(—R)—;

R, independently for each occurrence, represents H or lower alkyl;

R₁ represents one or more substituents, each independently selected from halogen, amino, acylamino, amidino, cyano, nitro, azido, ether, thioether, sulfoxido, -J-R₈, -J-OH, -J-lower alkyl, -J-lower alkenyl, -J-SH, -J-NH₂, or substituted or unsubstituted lower alkyl, lower alkenyl, cycloalkyl, heterocyclyl, cycloalkylalkyl, heterocyclylalkyl, aryl, heteroaryl, aralkyl, or heteroaralkyl, or protected forms of the above;

R₈, independently for each occurrence, represents H or substituted or unsubstituted lower alkyl, cycloalkyl, heterocyclyl, aralkyl, heteroaralkyl, aryl, or heteroaryl;

J represents, independently for each occurrence, a chain having 0-8 units selected from —C(—R)₂—, —N(—R)—, —O—, and —S—;

m is an integer from 0 to 2;

n is an integer from 0 to 2;

p is 0 or 1; and

q is an integer from 0 to 2, preferably 1,

and -Z-J-Hc, taken together, represent a substituted or unsubstituted nitrogen-containing heterocyclic or heteroaryl ring.

In certain embodiments, Hc is a substituted or unsubstituted five-membered ring, such as pyrrole, imidazole, triazole or pyridine.

Suitable substituents for Hc and Ar include halogen, cyano, alkyl (including perfluoroalkyl), alkenyl, alkynyl, aryl, hydroxyl, alkoxy, silyloxy, amino, nitro, thiol, amino, imino, amido, phosphoryl, phosphonate, carboxyl, carboxamide, silyl, thioether, alkylsulfonyl, arylsulfonyl, sulfoxide, selenoether, ketone, aldehyde, ester, or —(CH₂)ₘR₈, where m is an integer from 0 to 4. In certain embodiments, non-hydrogen substituents are selected from halogen, cyano, alkyl (including perfluoroalkyl), hydroxyl, alkoxy, alkenyl, alkynyl, aryl, nitro, thiol, imino, amido, carboxyl, thioether, alkylsulfonyl, arylsulfonyl, ketone, aldehyde, and ester. In certain embodiments, non-hydrogen substituents are selected from halogen, cyano, alkyl (including perfluoroalkyl), alkenyl, alkynyl, nitro, amido, carboxyl, alkylsulfonyl, ketone, aldehyde, and ester. Ar may, of course, be a bicyclic ring system, e.g., including two or more interconnected rings of which at least one ring is aromatic.

In certain embodiments, J represents, independently for each occurrence, a chain having from 0-4 (even more preferably 0-2) units selected from —C(—R)₂—, —N(—R)—, —O—, and —S—. In certain preferred embodiments, J represents substituted or unsubstituted methylene or ethylene units.

In certain embodiments, Y adjacent to Ar represents —O— or —S—, and preferably —O—. In certain embodiments, Z represents —N(—R)—, preferably —N(H)— or —N(—CH₂)—, or taken together with J and Hc represents a heterocyclic ring attached to the core via a nitrogen atom. In embodiments where Z-J-Hc taken together represent a heterocyclic ring, the ring may be, for example, a substituted or unsubstituted piperidine, piperazine, or pyrrolidine ring. For example, Z-J-Hc may represent a piperazine ring attached to the core via one nitrogen atom, with an aralkyl, aryl, heteroaralkyl, or heteroaryl substituent attached to the second nitrogen atom.

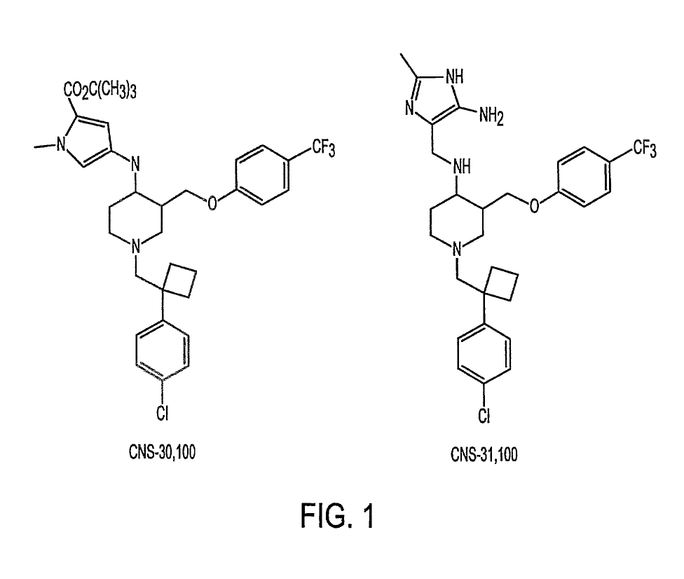

Certain representative illustrative DAT-5HT2 antagonists are shown in FIG. 1, including CNS-30,100 and CNS-31,100.

### Combinations Including DAT-5HT2 Agents

In certain embodiments, the method includes administering, conjointly with the pharmaceutical preparation, other agents intended to treat or prevent conditions associated with the anxiety, depression or psychotic disorder of interest. A drug to be administered conjointly with a subject DAT-5HT2 agent may be formulated together with the DAT-5HT2 agent as a single pharmaceutical preparation, e.g., as a pill or other medicament including both agents, or may be administered as a separate pharmaceutical preparation.

Exemplary combinations with the subject DAT-5HT2 agents include such other agents selected from a serotonin reuptake inhibitor, a 5HT₆ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, or a sedative-hypnotic drug.

Antidepressants

(i) Serotonin Reuptake Inhibitors (SRI).

The measurement of a compound's activity as an SSRI is now a standard pharmacological assay. Wong, et al., Neuropsychopharmacology 8, 337-344 (1993). Many compounds, including those discussed at length above, have such activity, and no doubt many more will be identified in the future. In the practice of the present invention, it is intended to include reuptake inhibitors which show 50% effective concentrations of about 1000 nM or less, in the protocol described by Wong supra. Serotonin reuptake inhibitors include, but are not limited to:

Fluoxetine, N-methyl-3-(p-trifluoromethylphenoxy)-3-phenylpropylami-ne, is marketed in the hydrochloride salt form, and as the racemic mixture of its two enantiomers. U.S. Pat. No. 4,314,081 is an early reference on the compound. Robertson et al., J. Med. Chem. 31, 1412 (1988), taught the separation of the R and S enantiomers of fluoxetine and showed that their activity as serotonin uptake inhibitors is similar to each other. In this document, the word “fluoxetine” will be used to mean any acid addition salt or the free base, and to include either the racemic mixture or either of the R and S enantiomers;

Duloxetine, N-methyl-3-(1-naphthalenyloxy)-3-(2-thienyl)propanamine-, is usually administered as the hydrochloride salt and as the (+) enantiomer. It was first taught by U.S. Pat. No. 4,956,388, which shows its high potency. The word “duloxetine” will be used here to refer to any acid addition salt or the free base of the molecule;

Venlafaxine is known in the literature, and its method of synthesis and its activity as an inhibitor of serotonin and norepinephrine uptake are taught by U.S. Pat. No. 4,761,501. Venlafaxine is identified as compound A in that patent;

Milnacipran (N,N-diethyl-2-aminomethyl-1-phenylcyclopropanecarboxamide) is taught by U.S. Pat. No. 4,478,836, which prepared milnacipran as its Example 4. The patent describes its compounds as antidepressants. Moret et al., Neuropharmacology 24, 1211-19 (1985), describe its pharmacological activities as an inhibitor of serotonin and norepinephrine reuptake;

Citalopram, 1-[3-(dimethylamino)propyl]-1-(4-fluorophenyl)-1,3-dihy-dro-5-isobenzofurancarbonitrile, is disclosed in U.S. Pat. No. 4,136,193 as a serotonin reuptake inhibitor. Its pharmacology was disclosed by Christensen et al., Eur. J. Pharmacol. 41, 153 (1977), and reports of its clinical effectiveness in depression may be found in Dufour et al., Int. Clin. Psychopharmacol. 2, 225 (1987), and Timmerman et al., ibid., 239;

Fluvoxamine, 5-methoxy-1-[4-(trifluoromethyl)-phenyl]-1-pentanone O-(2-aminoethyl)oxime, is taught by U.S. Pat. No. 4,085,225. Scientific articles about the drug have been published by Claassen et al., Brit. J. Pharmacol. 60, 505 (1977); and De Wilde et al., J. Affective Disord. 4, 249 (1982); and Benfield et al., Drugs 32, 313 (1986);

Paroxetine, trans-(−)-3-[(1,3-benzodioxol-5-yloxy)methyl]-4-(4-fluorophenyl)piperidine, may be found in U.S. Pat. Nos. 3,912,743 and 4,007,196. Reports of the drug's activity are in Lassen, Eur. J. Pharmacol. 47, 351 (1978); Hassan et al., Brit. J. Clin. Pharmacol. 19, 705 (1985); Laursen et al., Acta Psychiat. Scand. 71, 249 (1985); and Battegay et al., Neuropsychobiology 13, 31 (1985);

Sertraline, (1S-cis)-4-(3,4-dichlorophenyl)-1,2,3,4-tetrahydro-N-me-thyl-1-naphthylamine hydrochloride, is a serotonin reuptake inhibitor which is marketed as an antidepressant. It is disclosed by U.S. Pat. No. 4,536,518;

To illustrate, the SRI can be venlafaxine or a derivative thereof. For instance, the SRI can be a compound represented in the following formula, or a pharmaceutically acceptable salts thereof:

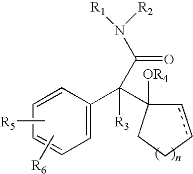

wherein

R₁ is hydrogen or alkyl of 1 to 6 carbon atoms;

R₂ is alkyl of 1 to 6 carbon atoms;

R₃ is hydrogen or alkyl of 1 to 6 carbon atoms;

R₄ is hydrogen, alkyl of 1 to 6 carbon atoms, formyl, or alkanoyl of 2 to 7 carbon atoms;

R₅ and R₆ are independently hydrogen, hydroxyl, alkyl of 1 to 6 carbon atoms, alkoxy of 1 to 6 carbon atoms, alkanoyloxy of 2 to 7 carbon atoms, cyano, nitro, alkylmercapto of 1 to 6 carbon atoms, amino, alkylamino of 1 to 6 carbon atoms, dialkylamino in which each alkyl group is of 1 to 6 carbon atoms, alkanamido of 2 to 7 carbon atoms, halo, trifluoromethyl, or, when taken together, methylene dioxy; and

n is one of the integers 0, 1, 2, 3 or 4.

The nontricyclic compound venlafaxine, chemically named (±)-1-[2-(dimethylamino)-1-(4-methoxyphenyl)ethyl]-cyclohexanol, is an antidepressant which has been studied extensively and which is described in, for example, U.S. Pat. No. 4,761,501 and Pento, J. T. Drugs of the Future 13(9):839-840 (1988).

Venlafaxine includes active derivatives of venlafaxine. The term “derivative” includes metabolites. Venlafaxine derivatives include: O-desmethylvenlafaxine and the single enantiomers of the two compounds.

In certain preferred embodiments, the venlafaxine compound is provided in optically pure form, such as optically pure (−)-N-desmethylvenlafaxine, chemically named (−)-1-[2-(methylamino)-1-(4-methoxyphenyl)ethyl]cyclohexanol; optically pure (−)-N,N-didesmethylvenlafaxine, chemically named (−)-1-[2-(amino)-1-(4-methoxyphenyl)ethyl]cyclohexanol; optically pure (−)-O-desmethylvenlafaxine, chemically named (−)-1-[2-(dimethylamino)-1-(4-phenol)ethyl]cyclohexanol; optically pure (−)-N,O-didesmethylvenlafaxine, chemically named (−)-1-[2-(methylamino)-1-(4phenol)ethyl]cyclohexanol; and optically pure (−)-O-desmethyl-N,N-didesmethylvenlafaxine, chemically named chemically named (−)-1-[2-(amino)-1-(4-phenol)ethyl]cyclohexanol.

In other embodiments, the SRI compound is optically pure a derivative of (+)-venlafaxine, such as (+)-O-desmethylvenlafaxine. U.S. Pat. No. 6,197,828 provides additional examples of derivatives of (+)-venlafaxine.

In preferred embodiments, the SRI is a selective serotonin reuptake inhibitor (SSRI). SSRIs include fluoxetinoids, sertraline (ZOLOFT), citalopram (CELEXA), paroxetine (PAXIL), and fluvoxamine (LUVOX), cericlamine, femoxetine, ifoxetine, cyanodothiepin, and litoxetine. The terms such as “sertraline,” “citalopram,” “paroxetine,” and “fluvoxamine” include active derivatives and metabolites, such as the demethyl metabolites norfluoxetine, demethylsertraline, and demethylcitalopram.

Preferred SSRIs are fluoxetinoids and citalopram (and its derivatives). More preferred SSRIs are fluoxetinoids.

Fluoxetinoids useful in the present methods and compositions include compounds that inhibit serotonin reuptake and have structures of the following formula:

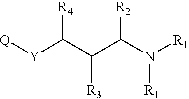

wherein, as valence and stability permit,

R₁, independently for each occurrence, represents H or lower alkyl, preferably H or Me;

R₂, R₃, and R₄ each independently represent H, methyl, substituted or unsubstituted phenyl, or substituted or unsubstituted phenylmethyl, such that exactly one of R₂, R₃, and R₄ is a substituted or unsubstituted phenyl, or substituted or unsubstituted phenylmethyl;

Y represents O, S, or —S(O)₂—, preferably O;

Q represents a substituted or unsubstituted aryl or heteroaryl ring, including polycyclic ring systems.

In certain embodiments, at least one occurrence of R₁ represents hydrogen.

In certain embodiments, R₂ and R₃ are selected from H and Me, preferably H, and R₄ represents a substituted or unsubstituted phenyl ring.

In certain embodiments, Q is a substituted or unsubstituted phenyl ring.

Examples of compounds which fall within the above formula can be found in U.S. Pat. Nos. 4,902,710, 4,824,868, 4,692,469, 4,626,549, 4,584,404 and 4,314,081.

In certain embodiments, a fluoxetinoid has a structure of the following formula:

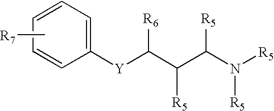

wherein, as valence and stability permit,

R₅, independently for each occurrence, represent H or Me;

R₆ represents a substituted or unsubstituted phenyl ring, preferably unsubstituted;

Y represents O, S, or —S(O)₂—, preferably O; and

R₇ represents from 1-5 substituents selected from halogen, lower alkyl, lower alkenyl, lower alkoxy, substituted or unsubstituted phenyl, and CF₃.

In certain embodiments, at least one occurrence of R₅ bound to N is a hydrogen.

In certain embodiments, R₆ represents an unsubstituted phenyl group.

In certain embodiments, R₇ represents from 1-2 substituents selected from halogen and CF₃.

Fluoxetine is metabolized far more slowly, with the primary metabolic derivative being norfluoxetine, which is similar to fluoxetine in selectivity and potency. Any combination of these compounds, racemic or enriched for either enantiomer, and pharmaceutically acceptable salts thereof may be employed in the methods and compositions described herein, and any one of these compounds is included in the term ‘fluoxetinoids’ as the term is used herein.

In certain embodiments, the SSRI is sertraline or a derivative thereof. For instance, the SSRI can be a compound represented in the following formula, or a pharmaceutically acceptable salt thereof:

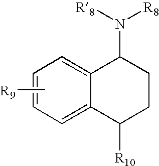

wherein

R₈ is selected from the group consisting of hydrogen and normal alkyl of from 1 to 3 carbon atoms;

R′₈ is normal alkyl of from 1 to 3 carbon atoms;

R₉ is selected from the group consisting of hydrogen, fluoro, chloro, bromo, trifluoromethyl and alkoxy of from 1 to 3 carbon atoms;

R₁₀ is

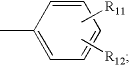

R₁₁ and R₁₂ are each independently selected from the group consisting of hydrogen, fluoro, chloro, bromo, trifluoromethyl, alkoxy of from 1 to 3 carbon atoms and cyano, with at least one of R₁₁ and R₁₂ being other than hydrogen; and

U.S. Pat. Nos. 4,536,518, 4,940,731, 4,962,128, and 5,130,338 describe sertraline and various derivatives and formulations thereof which can be used in the subject formulation and methods. Sertraline derivatives include N-desmethylsertraline.

In certain preferred embodiments, the compound is, as appropriate, the cis-isomeric base of the above formula. The term “cis-isomeric” refers to the relative orientation of the N(R′₈)R₈ and R₁₀ moieties on the cyclohexene ring (i.e. they are both oriented on the same side of the ring). Because both the 1- and 4-carbons of the formula are asymmetrically substituted, each cis-compound has two optically active enantiomeric forms denoted (with reference to the 1-carbon) as the cis-(1R) and cis-(1S) enantiomers. The preferred embodiment is the (1S) enantiomer, e.g., cis-(1S)—N-methyl-4-(3,4-dichlorophenyl)-1,2,3,4-tetrahydro-1-naphthalenamine and its pharmaceutically acceptable acid addition salts.

In certain embodiments, the SSRI is paroxetine or a derivative thereof. For instance, the SSRI can be a compound represented in the following formula, or a pharmaceutically acceptable salt thereof:

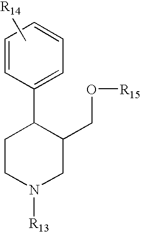

wherein

R₁₃ represents hydrogen or an alkyl group of 1-4 carbon atoms, and

R₁₄ represents hydrogen, alkyl having 1-4 carbon atoms, C1-6 alkoxy, C1-6 trifluoroalkyl (preferably, trifluoromethyl), hydroxy, halogen, methylthio, or C1-6 aryl(C1-6) alkyloxy (e.g., phenyl(C1-6)alkyloxy and benzyl(C1-6)alkyloxy), and

R₁₅ represents an alkyl or alkynyl group having 1-4 carbon atoms, or a phenyl group optionally substituted by C1-4 alkyl, C1-6 alkylthio, C1-6 alkoxy, halogen, nitro, acylamino, methylsulfonyl or methylenedioxy, or represents tetrahydronaphthyl.

In certain preferred embodiments, the SSRI is a compound represented in the following formula, or a pharmaceutically acceptable salt thereof:

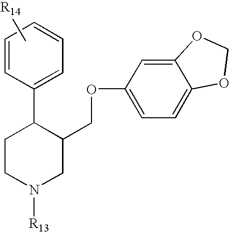

wherein R₁₃ represents hydrogen or an alkyl group of 1-4 carbon atoms, and R₁₄ is a halogen. In certain preferred embodiments, R₁₃ is a fluorine. Of particularly therapeutical effect is the (−) form of a compound of formula I, wherein R¹ is hydrogen and the fluorine is in para position.

The synthesis of paroxetine and of the acid addition salts thereof is described, inter alia, in U.S. Pat. No. 4,007,196 to Christensen et al. and U.S. Pat. No. 4,721,723 to Barnes et al. Derivative of paroxetine are also described in PCT publication WO035910.

In still other embodiments, the SSRI is citalopram or a derivative thereof. For instance, the SSRI can be a compound represented in the following formula, or a pharmaceutically acceptable salt thereof:

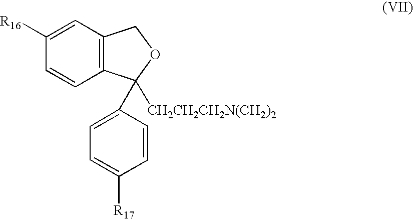

wherein R₁₆ and R₁₇ are each independently represent a halogen, a trifluoromethyl group, a cyano group or —C(═O)—R₁₈, wherein R₁₈ is an alkyl radical with from 1-4 C-atoms inclusive.

Citalopram was first disclosed in DE 2,657,271 corresponding to U.S. Pat. No. 4,136,193. This patent publication describes the preparation of citalopram by one method and outlines a further method which may be used for preparing citalopram Methods of preparing the individual enantiomers of citalopram are disclosed in U.S. Pat. No. 4,943,590, such as (+)-1-(3-Dimethylaminopropyl)-1-(4′-fluorophenyl)-1,3-dihydroisobenzofuran-5-carbonitrile. Citalopram derivatives include desmethylcitalopram and didesmethylcitalopram, and the single enantiomers of all three compounds.

In yet another embodiment, the SSRI is fluvoxamine or a derivative thereof. For instance, the SSRI can be a compound represented in the following formula, or a pharmaceutically acceptable salt thereof:

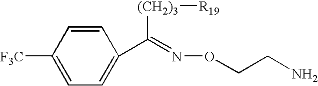

wherein R₁₉ represents a cyano group, a cyanomethyl group, a methoxymethyl group or an ethoxymethyl group. Fluvoxamine and other oxime ethers are disclosed in U.S. Pat. No. 4,085,225.

(ii) 5-HT₆ Receptor Antagonists

The subject DAT-5HT2 antagonists can be combined with 5-HT₆ receptor antagonists, such as bicyclic piperazinylbenzenesulfonamide, substituted N-phenyl-4-methoxy-3-piperazin-1-ylbenzenesulfonamides and conformationally restricted analogs. Exemplary 5-HT₆ receptor antagonists include:
(4-piperazin-1-ylquinolin-6-yl) arylsulfonamides such as described in Bromidge et al. Bioorg Med Chem Lett. 2001 Nov. 5; 11(21):2843-6.
5-Chloro-N-(4-methoxy-3-piperazin-1-yl-phenyl)-3-methyl-2-benzothiophenesulfonamide (SB-271046)
N-(2,5-dibromo-3-fluorophenyl)-4-methoxy-3-piperazin-1-ylbenzenesulfonamide (SB-357134)
4-amino-N-(2,6-bis-methylamino-pyrimidin-4-yl)-benzenesulfonamide (Ro 04-6790)

(iii) Miscellaneous

Suitable norepinephrine reuptake inhibitors of use in conjunction with the present invention include tertiary amine tricyclics and secondary amine tricyclics. Suitable examples of tertiary amine tricyclics include: amitriptyline, clomipramine, doxepin, imipramine and trimipramine, and pharmaceutically acceptable salts thereof. Suitable examples of secondary amine tricyclics include: amoxapine, desipramine, maprotiline, nortriptyline and protriptyline, and pharmaceutically acceptable salts thereof. Another norepinephrine reuptake inhibitor of use in conjunction with the present invention is reboxetine.

Suitable monoamine oxidase inhibitors of use in conjunction with the present invention include: isocarboxazid, phenelzine, tranylcypromine and selegiline, and pharmaceutically acceptable salts thereof. Suitable reversible inhibitors of monoamine oxidase of use in conjunction with the present invention include: moclobemide, and pharmaceutically acceptable salts thereof.

Suitable CRF antagonists of use in conjunction with the present invention include those compounds described in International Patent Specification Nos. WO 94/13643, WO 94/13644, WO 94/13661, WO 94/13676 and WO 94/13677.

Other antidepressants of use in conjunction with the present invention include adinazolam, alaproclate, amineptine, amitriptyline/chlordiazepoxide combination, atipamezole, azamianserin, bazinaprine, befuraline, bifemelane, binodaline, bipenamol, brofaromine bupropion, caroxazone, cericlamine, cianopramine, cimoxatone, citalopram, clemeprol, clovoxamine, dazepinil, deanol, demexiptiline, dibenzepin, dothiepin, droxidopa, enefexine, estazolam, etoperidone, femoxetine, fengabine, fezolamine, fluotracen, idazoxan, indalpine, indeloxazine, iprindole, levoprotiline, litoxetine, lofepramine, medifoxamine, metaprarine, metralindole, mianserin, milnacipran, minaprine, mirtazapine, montirelin, nebracetam, nefopam, nialamide, nomifensine, norfluoxetine, orotirelin, oxaflozane, pinazepam, pirlindone, pizotyline, ritanserin, sercloremine, setiptiline, sibutramine, sulbutiamine, sulpiride, teniloxazine, thozalinone, thymoliberin, tianeptine, tiflucarbine, tofenacin, tofisopam, toloxatone, tomoxetine, veralipride, viqualine, zimelidine and zometapine, and pharmaceutically acceptable salts thereof, and St. John's wort herb, or Hypericum perforatum, or extracts thereof.

Suitable classes of anti-anxiety agent of use in conjunction with the present invention also include benzodiazepines. Suitable benzodiazepines of use in conjunction with the present invention include: alprazolam, chlordiazepoxide, clonazepam, chlorazepate, diazepam, halazepam, lorazepam, oxazepam and prazepam, and pharmaceutically acceptable salts thereof.

In addition to benzodiazepines, other suitable classes of anti-anxiety agent are nonbenzodiazepine sedative-hypnotic drugs such as zolpidem; mood-stabilizing drugs such as clobazam, gabapentin, lamotrigine, loreclezole, oxcarbamazepine, stiripentol and vigabatrin; and barbiturates.

Suitable 5-HT_{1A} receptor agonists or antagonists of use in conjunction with the present invention include, in particular, the 5-HT_{1A} receptor partial agonists buspirone, flesinoxan, gepirone and ipsapirone, and pharmaceutically acceptable salts thereof. An example of a compound with 5-HT_{1A} receptor antagonist/partial agonist activity is pindolol.

Suitable CRF antagonists of use in conjunction with the present invention include those compounds described in International Patent Specification Nos. WO 94/13643, WO 94/13644, WO 94/13661, WO 94/13676 and WO 94/13677.

Another class of anti-anxiety agent of use in conjunction with the present invention are compounds having muscarinic cholinergic activity. Suitable compounds in this class include muscarinic cholinergic receptor agonists such as those compounds described in European Patent Specification Nos. 0709093, 0709094 and 0773021, and PCT Publication WO 96/12711.

Another class of anti-anxiety agent of use in conjunction with the present invention are compounds acting on ion channels. Suitable compounds in this class include carbamazepine, lamotrigine and valproate, and pharmaceutically acceptable salts thereof.

Anticonvulsants/Antiepileptics

Antiepileptic and anticonvulsants contemplated as the second component include, but are not limited to, phenyloins (phenyloin, mephenyloin and ethotoin), barbiturates (phenobarbital, mephobarbital, and primidone), iminostilbenes (carbamazepine), succinimides (ethosuximide), valproic acid, oxazolidinediones (trimethadione) and other antiseizure agents (gabapentin, lamotrigine, acetazolamide, felbamate, and γ-vinyl GABA).
Carbamezepine, 5H-dibenz[b,f]azepine-5-carboxamide is an anticonvulsant and analgesic marketed for trigeminal neuralgia; U.S. Pat. No. 2,948,718 (herein incorporated by reference in their entirety), discloses carbamezepine.
Valproic Acid, 2-propylpentanoic acid or dispropylacetic acid is a well known antiepileptic agent which dissociates to the valproate ion in the gastrointestinal tract; various pharmaceutically acceptable salts are disclosed in U.S. Pat. No. 4,699,927.
Lamotrigine, 6-(2,3-dichlorophenyl)-1,2,4-trizine-3,5-diamine is an antiepileptic drug indicated as adjunctive therapy in the treatment of partial seizures in adults with epilepsy. Lamotrigine is disclosed in U.S. Pat. No. 4,486,354.
Gabapentin, 1-(aminomethyl)cyclohexane acetic acid, is an anticonvulsant indicated as adjunctive therapy in the treatment of partial seizures with and without secondary generalization in adults with epilepsy. Gabapentin is described in U.S. Pat. Nos. 4,024,175 and 4,087,544.
Topiramate, 2,3:4,5-di-O-(1-isopropylidine)-3-D-fructopyranose sulphamate is an antiepileptic and disclosed in U.S. Pat. No. 4,513,006.

Atypical Antipsychotics

Another aspect of the invention relates to conjoint therapy using one or more of the subject DAT-5HT2 antagonists with an atypical antipsychotic, for the treatment of depression, anxiety, or a psychotic condition.

The essential feature of an atypical antipsychotic is less acute extrapyramidal symptoms, especially dystonias, associated with therapy as compared to a typical antipsychotic such as haloperidol. While conventional antipsychotics are characterized principally by D2 dopamine receptor blockade, atypical antipsychotics show antagonist effects on multiple receptors including the 5HT2a and 5HT2c receptors and varying degrees of receptor affinities. See Meltzer in Neuropsychopharmacology: The Fifth Generation of Progress, 2002, pp 819-831; and Baldessarini and Tarazi in Goodman & Gilman's The Pharmacological Basis of Therapeutics 10th Edition, 2001, p 485. Atypical antipsychotic drugs are also commonly referred to as serotonin/dopamine antagonists, reflecting the influential hypothesis that greater affinity for the 5HT2 receptor than for the 1)₂ receptor underlies “atypical” antipsychotic drug action or “second generation antipsychotic” drugs.

Clozapine, the prototypical atypical antipsychotic, differs from the typical antipsychotics with the following characteristics: (1) greater efficacy in the treatment of overall psychopathology in patients with schizophrenia nonresponsive to typical antipsychotics; (2) greater efficacy in the treatment of negative symptoms of schizophrenia; and (3) less frequent and quantitatively smaller increases in serum prolactin concentrations associated with therapy (Beasley, et al., Neuropsychopharmacology, 14(2), 111-123, (1996)). Atypical antipsychotics include, but are not limited to:
Olanzapine, 2-methyl-4-(4-methyl-1-piperazinyl)-10H-thieno[2,3-b][1-,5]benzodiazepine, is a known compound and is described in U.S. Pat. No. 5,229,382;
Clozapine, 8-chloro-11-(4-methyl-1-piperazinyl)-5H-dibenzo[b,e][1,4-]diazepine, is described in U.S. Pat. No. 3,539,573;
Risperidone, 3-[2-[4-(6-fluoro-1,2-benzisoxazol-3-yl)piperidino]ethyl]-2-methyl-6,7,8,9-tetrahydro-4H-pyrido-[1,2-a]pyrimidin-4-one, and its use in the treatment of psychotic diseases are described in U.S. Pat. No. 4,804,663;
Sertindole, 1-[2-[4-[5-chloro-1-(4-fluorophenyl)-1H-indol-3-yl]-1-piperidinyl]ethyl]imidazolidin-2-one, is described in U.S. Pat. No. 4,710,500. Its use in the treatment of schizophrenia is described in U.S. Pat. Nos. 5,112,838 and 5,238,945;
Quetiapine, 5-[2-(4-dibenzo[b,f][1,4]thiazepin-11-yl-1-piperazinyl)-ethoxy]ethanol, and its activity in assays which demonstrate utility in the treatment of schizophrenia are described in U.S. Pat. No. 4,879,288. Certain preferred embodiments, Quetiapine is provided as its (E)-2-butenedioate (2:1) salt; and
Ziprasidone, 5-[2-[4-(1,2-benzoisothiazol-3-yl)-1-piperazinyl]ethyl-]-6-chloro-1,3-dihydro-2H-indol-2-one, and especially its hydrochloride monohydrate. The compound is described in U.S. Pat. Nos. 4,831,031 and 5,312,925.

### E. Formulations

In another aspect, the present invention provides pharmaceutical preparations comprising the subject DAT-5HT2 antagonists. The DAT-5HT2 antagonists for use in the subject method may be conveniently formulated for administration with a biologically acceptable, non-pyrogenic, and/or sterile medium, such as water, buffered saline, polyol (for example, glycerol, propylene glycol, liquid polyethylene glycol, and the like) or suitable mixtures thereof. The optimum concentration of the active ingredient(s) in the chosen medium can be determined empirically according to procedures well known to behavioral scientists. As used herein, “biologically acceptable medium” includes any and all solvents, dispersion media, and the like which may be appropriate for the desired route of administration of the pharmaceutical preparation. The use of such media for pharmaceutically active substances is known in the art. Except insofar as any conventional media or agent is incompatible with the activity of the DAT-5HT2 antagonists, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations.”

Methods of introduction may also be provided by rechargeable or biodegradable devices. Various slow release polymeric devices have been developed and tested in vivo in recent years for the controlled delivery of drugs. A variety of biocompatible polymers (including hydrogels), including both biodegradable and non-degradable polymers, can be used to form an implant for the sustained release of an DAT-5HT2 antagonist at a particular target site. In accordance with the practice of this invention, it has been found that a dosage form and a method can be provided that administers an DAT-5HT2 antagonist in a program that substantially lessens or completely compensates for tolerance in a patient. Tolerance, as defined in Pharmacology in Medicine, by Brill, p. 227 (1965) McGraw-Hill, is characterized as a decrease in effect followed by administering a drug. When tolerance develops following a single dose or a few doses over a very short time, it is referred to as acute tolerance. When the drug is administered over a more protracted period of time to show a demonstrable degree of tolerance, it is referred to as chronic tolerance. The medical literature, as exemplified in The Pharmacological Bases of Therapeutics, by Goodman and Gilman, 8th Ed., p. 72 (1990) Pergamon Press, reported tolerance may be acquired to the effects of many drugs and this literature classifies tolerance as acute or chronic based on when it is acquired. That is, acute tolerance develops during a dosing phase of one dose or on one day, and chronic tolerance is acquired due to chronic administration, typically weeks, months, and years.

In certain embodiments, particularly where the selected DAT-5HT2 antagonist is one which may produce tolerance, e.g., acute tolerance, in the patient, it may be desirable to formulate the compound for variable dosing, and preferably for use in a dose-escalation regimen. In preferred embodiments, the subject DAT-5HT2 antagonists are formulated to deliver a sustained and increasing dose, e.g., over at least 4 hours, and more preferably, over at least 8 or even 16 hours.

In certain embodiments, representative dosage forms include hydrogel matrix containing a plurality of tiny pills. The hydrogel matrix comprises a hydrophilic polymer, such as a polysaccharide, agar, agarose, natural gum, alkali alginate including sodium alginate, carrageenan, fucoidan, furcellaran, laminaran, hypnea, gum arabic, gum ghatti, gum karaya, gum tragacanth, locust bean gum, pectin, amylopectin, gelatin, and a hydrophilic colloid. The hydrogel matrix comprises a plurality of tiny pills (such as 4 to 50), each tiny pill comprising an increasing dose population of from 100 ng ascending in dose, such as 0.5 mg, 1 mg, 1.2 mg, 1.4 mg, 1.6 mg, 1.8 mg, etc. The tiny pills comprise a release rate controlling wall of 0.0 mm to 10 mm thickness to provide for the timed ascending release of drug. Representative wall-forming materials include a triglyceryl ester selected from glyceryl tristearate, glyceryl monostearate, glyceryl dipalmitate, glyceryl laureate, glyceryl didecenoate, and glyceryl tridecenoate. Other wall forming materials comprise polyvinyl acetate phthalate, methylcellulose phthalate, and microporous vinyl olefins. Procedures for manufacturing tiny pills are disclosed in U.S. Pat. Nos. 4,434,153; 4,721,613; 4,853,229; 2,996,431; 3,139,383, and 4,752,470, which are incorporated by reference herein.

In certain embodiments, the drug releasing beads are characterized by a dissolution profile wherein 0 to 20% of the beads undergo dissolution and release the drug in 0 to 2 hours, 20 to 40% undergo dissolution and release the drug in 2 to 4 hours, 40 to 60% exhibit dissolution and release in 4 to 6 hours, 60 to 80% in 6 to 8 hours, and 80 to 100% in 8 to 10 hours. The drug releasing beads can include a central composition or core comprising a drug and pharmaceutically acceptable composition forming ingredients including a lubricant, antioxidant, and buffer. The beads comprise increasing doses of drug, for example, 1 mg, 2 mg, 5 mg, and so forth to a high dose, in certain preferred embodiments, of 15 to 100 mg. The beads are coated with a release rate controlling polymer that can be selected utilizing the dissolution profile disclosed above. The manufacture of the beads can be adapted from, for example, Liu et al. (1994) Inter. J. of Pharm., 112:105-116; Liu et al. (1994) Inter. J. of Pharm., 112:117-124; Pharm. Sci., by Remington, 14th Ed. pp. 1626-1628 (1970); Fincher et al. (1968) J. Pharm. Sci., 57:1825-1835; and U.S. Pat. No. 4,083,949.

Another exemplary dosage form provided by the invention comprises a concentration gradient of DAT-5HT2 antagonist from 1 mg to 15-600 mg coated from the former low dose to the latter high dose on a polymer substrate. The polymer can be an erodible or a nonerodible polymer. The coated substrate is rolled about itself from the latter high dose at the center of the dosage form, to the former low dose at the exposed outer end of the substrate. The coated substrate is rolled from the high dose to the low dose to provide for the release of from low to high dose as the substrate unrolls or erodes. For example, 1 mg to 600 mg of amphetamine is coated onto an erodible polymer such as an polypeptide, collagen, gelatin, or polyvinyl alcohol, and the substrate rolled concentrically from the high dose rolled over and inward to adapt a center position, and then outward towards the low dose to form an outer position. In operation, the dosage form erodes dispensing an ascending dose of amphetamine that is released over time.

Another dosage form provided by the invention comprises a multiplicity of layers, wherein each layer is characterized by an increasing dose of drug. The phrase “multiplicity of layers” denotes 2 to 6 layers in contacting lamination. The multiplicity of layers are positioned consecutively, that is, one layer after another in order, with a first exposed layer, the sixth layer in contact with the fifth layer and its exposed surface coated with a drug impermeable polymer. The sixth layer is coated with a drug impermeable polymer to insure release of the DAT-5HT2 antagonist from the first layer to the sixth layer. The first layer comprises, for example, 1 to 50 mg of drug and each successive layer comprises an additional 1 to 50 mg of drug. The biodegradable polymers undergo chemical decomposition to form soluble monomers or soluble polymer units. The biodegradation of polymers usually involves chemically or enzymatically catalyzed hydrolysis. Representative of biodegradable polymers acceptable for an increase drug loading in each layer of from 5 to 50 wt % over the first and successive layers wherein the first layer comprises 100 ng. Representative biodegradable polymers comprise biodegradable poly(amides), poly(amino acids), poly(esters), poly(lactic acid), poly(glycolic acid), poly(orthoesters), poly(anhydrides), biodegradable poly(dehydropyrans), and poly(dioxinones). The polymers are known to the art in Controlled Release of Drugs, by Rosoff, Ch. 2, pp. 53-95 (1989); and in U.S. Pat. Nos. 3,811,444; 3,962,414; 4,066,747; 4,070,347; 4,079,038; and 4,093,709.

In still other embodiments, the invention employs a dosage form comprising a polymer that releases a drug by diffusion, flux through pores, or by rupture of a polymer matrix. The drug delivery polymeric system comprises a concentration gradient, wherein the gradient is an ascent in concentration from a beginning or initial concentration to a final, or higher concentration. The dosage form comprises an exposed surface at the beginning dose and a distant nonexposed surface at the final dose. The nonexposed surface is coated with a pharmaceutically acceptable material impermeable to the passage of drug. The dosage form structure provides for a flux increase delivery of drug ascending from the beginning to the final delivered dose.

The dosage form matrix can be made by procedures known in the polymer art. In one manufacture, 3 to 5 or more casting compositions are independently prepared wherein each casting composition comprises an increasing dose of drug with each composition overlayered from a low to the high dose. This provides a series of layers that come together to provide a unit polymer matrix with a concentration gradient. In another manufacture, the higher dose is cast first followed by laminating with layers of decreasing dose to provide a polymer matrix with a drug concentration gradient. An example of providing a dosage form comprises blending a pharmaceutically acceptable carrier, like polyethylene glycol, with a known dose of an DAT-5HT2 antagonist and adding it to a silastic medical grade elastomer with a cross-linking agent, like stannous octanoate, followed by casting in a mold. The step is repeated for each successive layer. The system is allowed to set, e.g., for 1 hour, to provide the dosage form. Representative polymers for manufacturing the dosage form comprise olefin and vinyl polymers, condensation polymers, carbohydrate polymers, and silicon polymers as represented by poly(ethylene), poly(propylene), poly(vinyl acetate), poly(methyl acrylate), poly(isobutyl methacrylate), poly(alginate), poly(amide), and poly(silicone). The polymers and manufacturing procedures are known in Polymers, by Coleman et al., Vol. 31, pp. 1187-1230 (1990); Drug Carrier Systems, by Roerdink et al., Vol. 9, pp. 57-109 (1989); Adv. Drug Delivery Rev., by Leong et al., Vol. 1, pp. 199-233 (1987); Handbook of Common Polymers, compiled by Roff et al., (1971) published by CRC Press; and U.S. Pat. No. 3,992,518.

In still other embodiments, the subject formulations can be a mixture of different prodrug forms of one or more different DAT-5HT2 antagonists, each prodrug form having a different hydrolysis rate, and therefore activation rate, to provide an increasing serum concentration of the active DAT-5HT2 antagonists.

In other embodiments, the subject formulations can be a mixture of different DAT-5HT2 antagonists, each compound having a different rate of adsorption (such as across the gut or epithelia) and/or serum half-life.

The dose-escalation regimen of the present invention can be used to compensate for the loss of a therapeutic effect of an DAT-5HT2 antagonist, if any, by providing a method of delivery that continually compensates for the development of acute tolerance, by considering the clinical effect (E) of a drug at time (t) as a function of the drug concentration (C) according to Equation 1:

Effect=f(t,C)

In addition, the rate of drug delivered (A), in mg per hour, is inversely proportional to the concentration times the clearance of the drug. As the effect varies with time and the functionality is expressed, then, according to this invention, (A) can be governed to ensure the therapeutic effect is maintained at a clinical value. If the effect from a drug is found clinically to decrease with time, this decline could be linear as expressed by Equation 2:

Effect(t)=Effect(ini)−keffect*t

wherein, Effect(ini) is the clinical effect observed initially at the start of drug administration and Effect(t) is the effect observed at time (t) hours, keffect is a proportionality constant ascertained by measuring the clinical effect (E1) at time (t1) hours and (E2) at time (t2) hours while maintaining a constant plasma concentration followed by dividing (E1) minus (E2) by (t1) minus (t2). In order to maintain a constant effect, (A) must be adjusted with the same functionality according to Equation 3:

A(t)=A(ini)+keffect*t

wherein A(ini) is the initial drug input in mg per hour at the start of the therapy and A(t) is the drug input at time (t) hours, and keffect is the proportionality constant presented above. If the therapeutic effect is found to decline exponentially with time, this relationship is expressed by Equation 4:

Effect(t)=Effect(ini)*exp(−keffect*t)

wherein Effect(ini) and Effect(t) are as defined before, keffect is a rate constant (h⁻¹), a unit of reciprocal hours, ascertained by measuring the clinical effect (E1) at time (t1) hours and (E2) at time (t2) hours while maintaining a constant plasma concentration followed by dividing natural log of (E1) minus natural log of (E2) by (t1) minus (t2). To maintain a constant effect, (A) must be adjusted according to Equation 5:

A(t)=A(ini)*exp(keffect*t)

wherein A(ini) and A(t) are as defined before, keffect is the rate constant (h⁻¹) presented above. The equations are presented in Holford et al. (1982) Pharmac. Ther., 16:143-166.

The preparations of the present invention may be given orally, parenterally, topically, or rectally. They are of course given by forms suitable for each administration route. For example, they are administered in tablets or capsule form, by injection, infusion, inhalation, rectal suppository, or controlled release patch. Oral and controlled release patch administrations are preferred.

In certain preferred embodiments, the subject therapeutic is delivered by way of a transdermal patch. A patch is generally a flat hollow device with a permeable membrane on one side and also some form of adhesive to maintain the patch in place on the patient's skin, with the membrane in contact with the skin so that the medication can permeate out of the patch reservoir and into and through the skin. The outer side of the patch is formed of an impermeable layer of material, and the membrane side and the outer side are joined around the perimeter of the patch, forming a reservoir for the medication and carrier between the two layers.

Patch technology is based on the ability to hold an active ingredient in constant contact with the epidermis. Over substantial periods of time, drug molecules, held in such a state, will eventually find their way into the bloodstream. Thus, patch technology relies on the ability of the human body to pick up drug molecules through the skin. Transdermal drug delivery using patch technology has recently been applied for delivery of nicotine in an effort to assist smokers in quitting, the delivery of nitroglycerine to angina sufferers, the delivery of replacement hormones in post menopausal women, etc. These conventional drug delivery systems comprise a patch with an active ingredient such as a drug incorporated therein, the patch also including an adhesive for attachment to the skin so as to place the active ingredient in close proximity to the skin. Exemplary patch technologies are available from Ciba-Geigy Corporation and Alza Corporation. Such transdermal delivery devices can be readily adapted for use with the subject DAT-5HT2 antagonists.

The flux of the subject compounds across the skin can be modulated by changing either (a) the resistance (the diffusion coefficient), or (b) the driving force (the solubility of the drug in the stratum corneum and consequently the gradient for diffusion). Various methods can be used to increase skin permeation by the subject compounds, including penetration enhancers, use of pro-drug versions, superfluous vehicles, iontophoresis, phonophoresis, and thermophoresis. Many enhancer compositions have been developed to change one or both of these factors. See, for example, U.S. Pat. Nos. 4,006,218; 3,551,154; and 3,472,931, which respectively describe the use of dimethylsulfoxide (DMSO), dimethyl formamide (DMF), and N,N-dimethylacetamide (DMA) for enhancing the absorption of topically applied drugs through the stratum corneum. Combinations of enhancers consisting of diethylene glycol monoethyl or monomethyl ether with propylene glycol monolaurate and methyl laurate are disclosed in U.S. Pat. No. 4,973,468. A dual enhancer consisting of glycerol monolaurate and ethanol for the transdermal delivery of drugs is shown in U.S. Pat. No. 4,820,720. U.S. Pat. No. 5,006,342 lists numerous enhancers for transdermal drug administration consisting of fatty acid esters or fatty alcohol ethers of C2 to C4 alkanediols, where each fatty acid/alcohol portion of the ester/ether is of about 8 to 22 carbon atoms. U.S. Pat. No. 4,863,970 shows penetration-enhancing compositions for topical application comprising an active permeant contained in a penetration-enhancing vehicle containing specified amounts of one or more cell-envelope disordering compounds such as oleic acid, oleyl alcohol, and glycerol esters of oleic acid; a C2 or C3 alkanol; and an inert diluent such as water. Other examples are included in the teachings of U.S. Pat. No. 4,933,184 which discloses the use of menthol as a penetration enhancer; U.S. Pat. No. 5,229,130 which discloses the use of vegetable oil (soybean and/or coconut oil) as a penetration enhancer; and U.S. Pat. No. 4,440,777 which discloses the use of eucalyptol as a penetration enhancer.

The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration,” and “administered peripherally” as used herein mean the administration of a compound, drug, or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally, and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically acceptable dosage forms such as described below or by other conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration without being toxic to the patient.

The selected dosage level will depend upon a variety of factors including the activity of the particular compound of the present invention employed, or the ester, salt or amide thereof, the route of administration, the time of administration, the rate of excretion of the particular compound being employed, the duration of the treatment, other drugs, compounds and/or materials used in combination with the particular DAT-5HT2 antagonists employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, intravenous, intracerebroventricular, and subcutaneous doses of the compounds of this invention for a patient will range from about 0.0001 to about 100 mg per kilogram of body weight per day.

If desired, the effective daily dose of the active compound may be administered as two, three, four, five, six, or more sub-doses administered separately at appropriate intervals throughout the day, optionally, in unit dosage forms.

The term “treatment” is intended to encompass also prophylaxis, therapy, and cure.

The patient receiving this treatment is any animal in need, including primates, in particular, humans and other mammals such as equines, cattle, swine, and sheep; and poultry and pets in general.

The compound of the invention can be administered as such or in admixtures with pharmaceutically acceptable carriers and can also be administered in conjunction with other drugs such as dopamine precursors, dopaminergic agents, dopaminergic and anti-cholinergic agents, anti-cholinergic agents, dopamine agonists, MAO-B (monoamine oxidase B) inhibitors, COMT (catechol O-methyltransferase) inhibitors, muscle relaxants, sedatives, anticonvulsant agents, dopamine reuptake inhibitors, dopamine blockers, β-blockers, carbonic anhydrase inhibitors, narcotic agents, GABAergic agents, or alpha antagonists. Conjunctive therapy thus includes sequential, simultaneous and separate administration of the active compound in a way that the therapeutic effects of the first one administered are not entirely absent when the subsequent is administered.

While it is possible for a compound of the present invention to be administered alone, it is preferable to administer the compound as a pharmaceutical formulation (composition). The DAT-5HT2 antagonists according to the invention may be formulated for administration in any convenient way for use in human or veterinary medicine.

Thus, another aspect of the present invention provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, or pastes for application to the tongue; (2) parenteral administration, for example, by subcutaneous, intramuscular, or intravenous injection as, for example, a sterile solution or suspension; (3) topical application, for example, as a cream, ointment, or spray applied to the skin; or (4) intravaginally or intrarectally, for example, as a pessary, cream, or foam. However, in certain embodiments, the subject compounds may be simply dissolved or suspended in sterile water.

The phrase “pharmaceutically acceptable carrier” as used herein means a pharmaceutically acceptable material, composition, or vehicle, such as a liquid or solid filter, diluent, excipient, solvent, or encapsulating material, involved in carrying or transporting the subject regulators from one organ or portion of the body to another organ or portion of the body. Each carrier must be “acceptable” in the sense of being compatible with the other ingredients of the formulation and not injurious to the patient. Some examples of materials which can serve as pharmaceutically acceptable carriers include (1) sugars, such as lactose, glucose, and sucrose; (2) starches, such as corn starch and potato starch; (3) cellulose and its derivatives, such as sodium carboxymethyl cellulose, ethyl cellulose, and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil, and soybean oil; (10) glycols, such as propylene glycol; (11) polyols, such as glycerin, sorbitol, mannitol, and polyethylene glycol; (12) esters such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As set out above, certain embodiments of the present DAT-5HT2 antagonists may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid and isolating the salt thus formed. Representative salts include but are not limited to following: 2-hydroxyethanesulfonate, 2-naphthalenesulfonate, 3-hydroxy-2-naphthoate, 3-phenylpropionate, acetate, adipate, alginate, amsonate, aspartate, benzenesulfonate, benzoate, besylate, bicarbonate, bisulfate, bitartrate, borate, butyrate, calcium edetate, camphorate, camphorsulfonate, camsylate, carbonate, citrate, clavulariate, cyclopentanepropionate, digluconate, dodecylsulfate, edetate, edisylate, estolate, esylate, ethanesulfonate, fumarate, gluceptate, glucoheptanoate, gluconate, glutamate, glycerophosphate, glycollylarsanilate, hemisulfate, heptanoate, hexafluorophosphate, hexanoate, hexylresorcinate, hydrabamine, hydrobromide, hydrochloride, hydroiodide, hydroxynaphthoate, iodide, isothionate, lactate, lactobionate, laurate, laurylsulphonate, malate, maleate, mandelate, mesylate, methanesulfonate, methylbromide, methylnitrate, methylsulfate, mucate, naphthylate, napsylate, nicotinate, nitrate, N-methylglucamine ammonium salt, oleate, oxalate, palmitate, pamoate, pantothenate, pectinate, persulfate, phosphate, phosphate/diphosphate, picrate, pivalate, polygalacturonate, propionate, p-toluenesulfonate, salicylate, stearate, subacetate, succinate, sulfate, sulfosaliculate, suramate, tannate, tartrate, teoclate, thiocyanate, tosylate, triethiodide, undecanoate, and valerate salts, and the like. (See, for example, Berge et al. (1977) “Pharmaceutical Salts,” J. Pharm. Sci. 66:1-19)

In certain embodiments, the pharmaceutically acceptable salts of the subject compounds include the conventional non-toxic salts of the compounds, e.g., from non-toxic organic or inorganic acids. Particularly suitable are salts of weak acids. For example, such conventional non-toxic salts include those derived from inorganic acids such as hydrochloric, hydrobromic, hydriodic, cinnamic, gluconic, sulfuric, sulfamic, phosphoric, nitric, and the like; and the salts prepared from organic acids such as acetic, propionic, succinic, glycolic, stearic, lactic, maleic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the compounds of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of compounds of the present invention. These salts can likewise be prepared in situ during the final isolation and purification of the compounds, or by separately reacting the purified compound in its free acid form with a suitable base, such as the hydroxide, carbonate, or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts, and the like. Representative organic amines useful for the formation of base addition salts include ethylamine, diethylamine, ethylenediamine, ethanolamine, diethanolamine, piperazine, and the like. (See, for example, Berge et al., supra)

Wetting agents, emulsifiers, and lubricants, such as sodium lauryl sulfate and magnesium stearate, as well as coloring agents, release agents, coating agents, sweetening, flavoring and perfuming agents, preservatives, and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite, and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal, and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), and/or as mouth washes, and the like, each containing a predetermined amount of a compound of the present invention as an active ingredient. A compound of the present invention may also be administered as a bolus, electuary, or paste.

In solid dosage forms of the invention for oral administration (capsules, tablets, pills, dragees, powders, granules, and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets, and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), or surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes, and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents, and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, and sweetening, flavoring, coloring, perfuming, and preservative agents.

Suspensions, in addition to the active compounds, may contain suspending agents, such as ethoxylated isostearyl alcohols, polyoxyethylene sorbitol and sorbitan esters, microcrystalline cellulose, aluminum metahydroxide, bentonite, agar-agar, and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax, or a salicylate, and which is solid at room temperature, but liquid at body temperature and, therefore, will melt in the rectum or vaginal cavity and release the active DAT-5HT2 antagonist.

Formulations of the present invention which are suitable for vaginal administration also include pessaries, tampons, creams, gels, pastes, foams, or spray formulations containing such carriers as are known in the art to be appropriate.

Dosage forms for the topical or transdermal administration of a compound of this invention include powders, sprays, ointments, pastes, creams, lotions, gels, solutions, patches, and inhalants. The active compound may be mixed under sterile conditions with a pharmaceutically acceptable carrier, and with any preservatives, buffers, or propellants which may be required.

The ointments, pastes, creams, and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to a compound of this invention, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

In certain embodiments, the subject compound(s) are formulated as part of a transdermal patch. Transdermal patches have the added advantage of providing controlled delivery of a compound of the present invention to the body. Such dosage forms can be made by dissolving or dispersing the DAT-5HT2 antagonists in the proper medium. Absorption enhancers can also be used to increase the flux of the DAT-5HT2 antagonists across the skin. The rate of such flux can be controlled by either providing a rate-controlling membrane or dispersing the compound in a polymer matrix or gel.

The “free base form” of the subject compound relates to a form in which the compound is not complexed with an acid, e.g., is not an ammonium salt. Such forms may be incorporated into a patch. It will be appreciated that the DAT-5HT2 antagonists may be complexed, for example, with elements of the drug-retaining matrix of the patch and, as such, the DAT-5HT2 antagonists may not necessarily be in the form of the free base, when actually retained by the patch.

The patch preferably comprises a drug-impermeable backing layer. Suitable examples of drug-impermeable backing layers which may be used for transdermal or medicated patches include films or sheets of polyolefins, polyesters, polyurethanes, polyvinyl alcohols, polyvinyl chlorides, polyvinylidene chloride, polyamides, ethylene-vinyl acetate copolymer (EVA), ethylene-ethylacrylate copolymer (EEA), vinyl acetate-vinyl chloride copolymer, cellulose acetate, ethyl cellulose, metal vapour deposited films or sheets thereof, rubber sheets or films, expanded synthetic resin sheets or films, non-woven fabrics, fabrics, knitted fabrics, paper, and foils. Preferred drug-impermeable, elastic backing materials are selected from polyethylene tereplithalate (PET), polyurethane, ethylene-vinyl acetate copolymer (EVA), plasticized polyvinylchloride, and woven and non-woven fabric. Especially preferred is non-woven polyethylene tereplithalate (PET). Other backings will be readily apparent to those skilled in the art.

The term “block copolymer,” in the preferred adhesives of the invention, refers to a macromolecule comprised of two or more chemically dissimilar polymer structures, terminally connected together (Block Copolymers: Overview and Critical Survey, Noshay and McGrath, 1977). These dissimilar polymer structures, sections or segments, represent the “blocks” of the block copolymer. The blocks may generally be arranged in an A-B structure, an A-B-A structure, or a multi-block -(A-B)n- system, wherein A and B are the chemically distinct polymer segments of the block copolymer.

It is generally preferred that the block copolymer is of an A-B-A structure, especially wherein one of A and B is an acrylic-type polymeric unit. It will be appreciated that the present invention is also applicable using block copolymers which possess three or more different blocks, such as an A-B—C block copolymer. However, for convenience, reference hereinafter to block copolymers will assume that there are only A and B sub-units, but it will be appreciated that such reference also encompasses block copolymers having more than two different sub-units, unless otherwise specified.

It will be appreciated that the properties of block copolymers are very largely determined by the nature of the A and B blocks. Block copolymers commonly possess both ‘hard’ and ‘soft’ segments. A ‘hard’ segment is a polymer that has a glass transition temperature (Tg) and/or a melting temperature (Tm) that is above room temperature, while a ‘soft’ segment is a polymer that has a Tg (and possibly a Tm) below room temperature. The different segments are thought to impart different properties to the block copolymer. Without being constrained by theory, it is thought that association of the hard segments of separate block copolymer units result in physical cross-links within the block copolymer, thereby promoting cohesive properties of the block copolymer. It is particularly preferred that the hard segments of the block copolymers form such physical close associations.

The block copolymers useful in the present invention preferably are acrylic block copolymers. In acrylic block copolymers, at least one of the blocks of the block copolymer is an acrylic acid polymer or a polymer of an acrylic acid derivative. The polymer may be composed of just one repeated monomer species. However, it will be appreciated that a mixture of monomeric species may be used to form each of the blocks, so that a block may, in itself, be a copolymer. The use of a combination of different monomers can affect various properties of the resulting block copolymer. In particular, variation in the ratio or nature of the monomers used allows properties such as adhesion, tack, and cohesion to be modulated, so that it is generally advantageous for the soft segments of the block copolymer to be composed of more than one monomer species.

It is preferred that alkyl acrylates and alkyl methacrylates are polymerized to form the soft portion of the block copolymer. Alkyl acrylates and alkyl methacrylates are thought to provide properties of tack and adhesion. Suitable alkyl acrylates and alkyl methacrylates include n-butyl acrylate, n-butyl methacrylate, hexyl acrylate, 2-ethylbutyl acrylate, isooctyl acrylate, 2-ethylhexyl acrylate, 2-ethylhexyl methacrylate, decyl acrylate, decyl methacrylate, dodecyl acrylate, dodecyl methacrylate, tridecylacrylate, and tridecyl methacrylate, although other suitable acrylates and methacrylates will be readily apparent to those skilled in the art. It is preferred that the acrylic block copolymer comprises at least 50% by weight of alkyl acrylate or alkyl methacrylate(co)polymer.

Variation in the components of the soft segment affects the overall properties of the block copolymer, although the essential feature remains the cross-linking of the soft segments. For example, soft segments essentially consisting of diacetone acrylamide with either butyl acrylate and/or 2-ethylhexyl acrylate, in approximately equal proportions, work well, and a ratio by weight of about 3:4:4 provides good results. It is preferred that diacetone acrylamide or other polar monomer, such as hydroxyethylmethacrylate or vinyl acetate, be present in no more than 50% w/w of the monomeric mix of the soft segment, as this can lead to reduced adhesion, for example. The acrylate component may generally be varied more freely, with good results observed with both 2-ethylhexyl acrylate and butyl acrylate together or individually.

As noted above, ratios of the various monomers are generally preferred to be approximately equal. For adhesives, this is preferred to be with a polar component of 50% or less of the soft segment, with the apolar portion forming up to about 85% w/w, but preferably between about 50 and 70% w/w. In the example above, this is about 72% (4+4) polar to about 18% (3) polar.

In general, it is particularly preferred that any apolar monomer used does not confer acidity on the adhesive. Adhesives of the invention are preferably essentially neutral, avoiding any unnecessary degeneration of the DAT-5HT2 antagonists.

Limiting active functionalities, especially those with active hydrogen, is generally preferred, in order to permit wide use of any given formulation of adhesive without having to take into account how it is likely to interact chemically with its environment. Thus, a generally chemically inert adhesive is preferred, in the absence of requirements to the contrary.

As discussed above, polymers suitable for use as the hard portion of the block copolymer possess glass transition temperatures above room temperature. Suitable monomers for use in forming the hard segment polymer include styrene, x-methylstyrene, methyl methacrylate, and vinyl pyrrolidone, although other suitable monomers will be readily apparent to those skilled in the art. Styrene and polymethylmethacrylate have been found to be suitable for use in the formation of the hard segment of the block copolymers. It is preferred that the hard portion of the block copolymer forms from 3-30% w/w of the total block copolymer, particularly preferably from 5-15% w/w.

The block copolymer is further characterized in that the soft portions contain a degree of chemical cross-linking. Such cross-linking may be effected by any suitable cross-linking agent. It is particularly preferable that the cross-linking agent be in the form of a monomer suitable for incorporation into the soft segment during polymerization. Preferably the cross-linking agent has two or more radically polymerizable groups, such as a vinyl group, per molecule of the monomer, at least one tending to remain unchanged during the initial polymerization, thereby permitting cross-linking of the resulting block copolymer.

Suitable cross-linking agents for use in the present invention include divinylbenzene, methylene bis-acrylamide, ethylene glycol di(meth)acrylate, ethyleneglycol tetra(meth)acrylate, propylene glycol di(meth)acrylate, butylene glycoldi(meth)acrylate, or trimethylolpropane tri(meth)acrylate, although other suitable cross-linking agents will be readily apparent to those skilled in the art. A preferred cross-linking agent is tetraethylene glycol dimethacrylate. It is preferred that the cross-linking agent comprises about 0.01-0.6% by weight of the block copolymer, with 0.1-0.4% by weight being particularly preferred.

Methods for the production of block copolymers from their monomeric constituents are well known. The block copolymer portions of the present invention may be produced by any suitable method, such as step growth, anionic, cationic, and free radical methods (Block Copolymers, supra). Free radical methods are generally preferred over other methods, such as anionic polymerization, as the solvent and the monomer do not have to be purified.

Suitable initiators for polymerization include polymeric peroxides with more than one peroxide moiety per molecule. An appropriate choice of reaction conditions is well within the skill of one in the art, once a suitable initiator has been chosen.

The initiator is preferably used in an amount of 0.005-0.1% by weight of the block copolymer, with 0.01-0.05% by weight being particularly preferred, although it will be appreciated that the amount chosen is well within the skill of one in the art. In particular, it is preferred that the amount should not be so much as to cause instant gelling of the mix, nor so low as to slow down polymerization and to leave excess residual monomers. A preferred level of residual monomers is below 2000 ppm.

It will also be appreciated that the amount of initiator will vary substantially, depending on such considerations as the initiator itself and the nature of the monomers.

The block copolymers are adhesives, and preferably are pressure sensitive adhesives. Pressure sensitive adhesives can be applied to a surface by hand pressure and require no activation by heat, water, or solvent. As such, they are particularly suitable for use in accordance with the present invention.

The block copolymers may be used without tackifiers and, as such, are particularly advantageous. However, it will be appreciated that the block copolymers may also be used in combination with a tackifier, to provide improved tack, should one be required or desired. Suitable tackifiers are well known and will be readily apparent to those skilled in the art.

Without being constrained by theory, it is thought that the combination of chemical cross-links between the soft segments of the copolymer combined with the, generally, hydrophobic interaction, or physical cross-linking, between the hard portions results in a “matrix-like” structure. Copolymers having only physical cross-linking of the hard segments are less able to form such a matrix. It is believed that the combination of both forms of cross-linking of the block copolymers provides good internal strength (cohesion) and also high drug storage capacity.

More particularly, it is believed that the hard segments associate to form “islands,” or nodes, with the soft segments radiating from and between these nodes.

There is a defined physical structure in the “sea” between the islands, where the soft segments are cross-linked, so that there is no necessity for extensive intermingling of the soft segments. This results in a greater cohesion of the whole block copolymer while, at the same time, allowing shortened soft segment length and still having as great, or greater, distances between the islands, thereby permitting good drug storage capacity.

The block copolymer preferably cross-links as the solvent is removed, so that cross-linking can be timed to occur after coating, this being the preferred method.

Accordingly, not only can the block copolymer easily be coated onto a surface, but the complete solution can also be stored for a period before coating. Accordingly, in the manufacturing process of the patches, the process preferably comprises polymerizing the monomeric constituents of each soft segment in solution, then adding the constituents of the hard segment to each resulting solution and polymerizing the resulting mix, followed by cross-linking by removal of any solvent or solvent system, such as by evaporation. If the solution is to be stored for any length of time, it may be necessary to keep the polymer from precipitating out which may be achieved by known means, such as by suspending agents or shaking. It may also be necessary to select the type of polymers that will be subject to substantially no cross-linking until the solvent is evaporated.

In general, it is preferred that the adhesive possesses a minimum number of functionalities having active hydrogen, in order to avoid undesirable reactions/interactions, such as with any drug that it is desired to incorporate into the adhesive material. It will be appreciated that this is only a preferred restriction, and that any adhesive may be tailored by one skilled in the art to suit individual requirements.

Suitable monomers for use in forming the hard segment include styrene, a-methylstyrene, methyl methacrylate, and vinyl pyrrolidone, with the preferred proportion of the hard segment being between 5 and 15% w/w. In particular, it is advantageous to use the compounds of WO 99/02141, as it is possible to load over 30% of drug into such a system.

Thus, in the patches of the present invention, it is generally possible to calculate the amount of drug required and determine the appropriate patch size with a given drug loading in accordance with a patient's body weight which can be readily calculated by those skilled in the art.

In certain embodiments, small amounts of plasticizer, such as isopropyl myristate (IPM), are incorporated. This has the advantage of helping solubilize the DAT-5HT2 antagonist(s) as well as rendering the adhesive less rough on the skin. Levels of between 2 and 25%, by weight, are generally useful, with levels of between 3 and 20% being more preferred and levels of 5 to 15%, especially about 10%, being most preferred. Other plasticizers may also be used, and suitable plasticizers will be readily apparent to those skilled in the art.

Plasticizers generally take the form of oily substances introduced into the adhesive polymer. The effect of the introduction of such oily substances is to soften the physical structure of the adhesive whilst, at the same time, acting at the interface between the adhesive and the skin, thereby helping to somewhat weaken the adhesive, and to reduce exfoliation.

The free base oil may be obtained by basifying salts of the subject compounds, or any other suitable salt, with a suitable base, in the presence of a hydrophilic solvent, especially water, and an organic solvent. For instance, water and ethyl acetate, in approximately equal proportions, work well, with ammonia serving as the basifying agent. The water may then be removed and the preparation washed with further water, or other aqueous preparation, after which the preparation may be suitably extracted with ether, for example, after having removed the ethyl acetate. It is preferred to keep the preparation under an inert atmosphere, especially after completion.

Whilst it will be appreciated that patches of the present invention may be removed from the patient at any time once it is desired to terminate a given dose, this can have the disadvantage of providing an opportunity for potential drug abuse of the partially discharged patch. Abuse of the subject compounds is highly undesirable.

In certain embodiments, it may be advantage to use a patch tailored to have delivered, by about 8 hours after application, the majority of the subject compound that it is capable of delivering in a 24 hour period, so that a patch can be left in place, and levels of drug still diminish appreciably. It is advantageous that the drug delivery profile has first order kinetics, so that the majority of the drug is delivered during the main part of the day and, even if the patient omits to remove the patch, the amount of drug is moving towards exhaustion by the end of the day, and the amount of drug is dropping rapidly.

It will be appreciated that patches of the invention may be constructed in any suitable manner known in the art for the manufacture of transdermal patches. The patches may simply comprise adhesive, drug, and backing, or may be more complex, such as having edging to prevent seepage of drug out of the sides of the patch. Patches may also be multi-layered.

Ophthalmic formulations, eye ointments, powders, solutions, and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient, or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

### EXEMPLIFICATION

The invention now being generally described, it will be more readily understood by reference to the following examples which are included merely for purposes of illustration of certain aspects and embodiments of the present invention, and are not intended to limit the invention.

### Example 1

### In Vitro Profiles of Two Illustrative DAT-5HT2 Antagonists

In vitro inhibitory profiles of two illustrative DAT-5HT2 antagonists, CNS-30,100 and CNS-31,100, were determined by measuring their respective IC₅₀ using standard assays.

In a typical uptake assay for measuring IC50 of DAT, the assay is performed at room temperature in Krebs-Ringer's-HEPES (KRH) buffer (125 mM NaCl, 4.8 mM KCl, 1.2 mM MgSO4, 1.2 mM KH2PO4, 1.3 mM CaCl2, and 25 mM HEPES, pH 7.4), supplemented with 0.1% D-glucose, 1 mM ascorbic acid, 1 mM tropolone [catechol-O-methyltransferase (EC 2.1.1.6)-inhibitor] and 10 μM pargyline (monoamine oxidase-B inhibitor). Before the assay, cells expressing DAT are washed once with KRH and equilibrated for 5 min. The cells may be assayed in 24-well plates and incubated for 2-5 min with tritiated amines. Nontransported inhibitors were preincubated for 5 min, and substrates were applied together with the tritiated substrate. The uptake assay is terminated with two washes of ice-cold KRH, and the accumulated radioactivity is recovered by lysing the cells in 0.2% SDS and 0.1 N NaOH and counting on a Liquid Scintillation Analyzer 1900 TR (Packard, Meriden, Conn.). Nonspecific uptake can be determined in the presence of 10 μM GBR12909 (for hDAT).

Experiments to determine the ionic requirements for DAT-mediated uptake are done in KRH buffer, substituting LiCl or choline Cl for NaCl (sodium-dependence) or substituting D-gluconates for NaCl and KCl, and Ca(NO₃)₂ for CaCl₂ (chloride dependence). Cells are washed twice with sodium- or chloride-free KRH before the assay (each wash step at least 5 min). In all transport assays, incubation periods and substrate concentrations are chosen such that uptake obeyed first-order rate kinetics.

Vₘₐₓ values for amine uptake in stable transfected DAT-cells are determined in parallel assays for at least two amines per experiment and expressed as relative values.

Similarly, IC₅₀ for 5-HT-2A/C receptors can be measured using a standard uptake assay using labeled 5-HT. See Rudolph et al., J. Pharmacol. Exp. Ther. 287(1): 389-94, 1998. Briefly, the time course of the 5HT uptake can be determined by incubating each cell/tissue with the receptor at different periods in containers containing 2 ml of RBS without Ca++ at 30° C. in which [³H]5HT (38 nM) is added. For estimating the kinetic parameters of 5HT uptake, several concentrations of [³H]5HT may be used, ranging, for example, from 25 to 240 nM, using a 30-min incubation period. Corresponding incubations were conducted in a Na+-free medium to correct for nonspecific uptake. Radioactivity not incorporated by the tissue/cell is washed off by further incubation for 10 min in 100 ml of RBS. The tissue/cell is homogenized in 2 ml of 10% perchloric acid at the end of the incubation. The resulting suspension is centrifuged at 600×g for 5 min and 0.1 ml of the supernatant is analyzed for radioactivity.

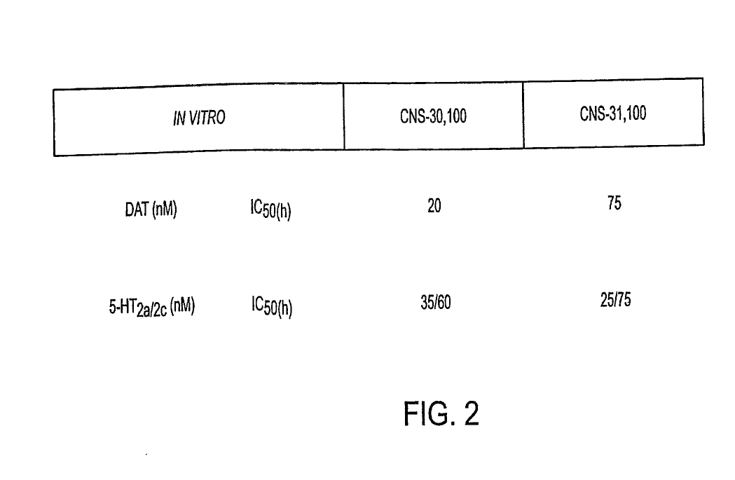

FIG. 2 represents a typical result in table form. Specifically, the IC₅₀ for CNS-30,100 against DAT (SLC6A3) is 20 nM, while its IC₅₀ for 5-HT-2A/2C receptors is about 35-60 nM.

Similar results were also obtained for CNS-31,100, where the IC₅₀ for DAT is about 75 nM, while its IC₅₀ for 5-HT-2A/2C receptors is about 25-75 nM.

These data demonstrate that the subject DAT-5HT2 antagonists inhibit both the intended targets, DAT and 5HT2 receptors.

### Example 2

### In Vivo Efficacy of Several Illustrative DAT-5HT2 Antagonists

In vivo efficacy of DAT-5HT2 antagonists of the instant invention can be measured using standard forced swim test model using rat.

A typical forced swim assay is described in Porsolt et al., Nature 266: 730-732, 1977; and Porsolt et al., in Psychopharmacology, Olivier, Mos, and Slangen (eds) Birkhauser Verlag, Basel, pp. 137-159, 1991. Briefly, when mice (or rats) are forced to swim in a cylinder from which no escape is possible, they readily adopt a characteristic immobile posture and make no further attempts to escape except for small movements needed to keep floating. The immobility is considered by some to reflect a “depressive mood” (Porsolt et al., Nature 266: 730-732, 1977) in which animals cease to struggle to escape the aversive situation. The immobility induced by the procedure is influenced by a wide variety of antidepressants (Porsolt et al., in Psychopharmacology, Olivier, Mos, and Slangen (eds) Birkhauser Verlag, Basel, pp. 137-159, 1991) and has a good predictive validity in that it detects antidepressants with different mechanisms of action (TCAs, SSRIs, MAOIs, and other atypical ones). The test is sensitive to muscle-relaxant (benzodiazepines) and sedative (neuroleptics) effects, leading to enhanced immobility (Porsolt et al., in Psychopharmacology, Olivier, Mos, and Slangen (eds) Birkhauser Verlag, Basel, pp. 137-159, 1991).

In a typical experiment, rats are placed singly into a cylinder (46×30 cm) containing fresh water at 23° C. for 6 minutes. The activity (or immobility) of the animal is measured by an observer minute by minute.

In order to measure the in vivo efficacy of inhibiting DAT and the 5-HT-2A/2C receptors in rats using the DAT-5HT2 antagonists of the instant invention, one test inhibitor (CNS-30,100 or CNS-31,100) is injected i.p. into the animals, at various doses (e.g. 7.5 and 15 mg/kg). Sibutramine, Bupropion, and Imipramine are similarly administered as controls. It is expected that DAT-5HT2 antagonists perform equally well, if not better, than the commercial drugs Sibutramine, Bupropion, and Imipramine.

Other administration routes may also be used for this experiment.

### Example 3

### Toxicological Profiles of Illustrative DAT-5HT2 Antagonists

To investigate the toxicological profile of the subject DAT-5HT2 antagonists, experimental rats in small groups (e.g. 5 animals/group), are administered with various doses of respective DAT-5HT2 antagonists (e.g. 30, 90, 120, and 200 mg/kg), and the observed toxicological effects are recorded.

It is expected that rats can tolerate doses below 90-120 mg/kg of the subject DAT-5HT2 antagonists, with no significant observed symptoms associated with drug administration. At higher doses, such as 200 mg/kg, animals may show decreased grip strength, and/or slight depression.

### EQUIVALENTS

Those skilled in the art will recognize, or be able to ascertain using no more than routine experimentation, many equivalents to the specific embodiments of the invention described herein. Such equivalents are intended to be encompassed by the following claims.

All patents, publications, and other references cited above are hereby incorporated by reference in their entirety.

What is claimed is:

1. A DAT-5HT2 antagonist represented by Formula I, or a pharmaceutically acceptable salt, solvate, metabolite or pro-drug thereof: 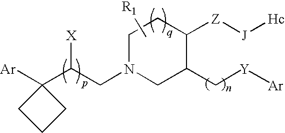
wherein, as valence and stability permit,
Ar, independently for each occurrence, represents a substituted or unsubstituted aryl or heteroaryl ring;
Hc represents a substituted or unsubstituted nitrogen-containing heteroaryl ring;
X represents H or OR;
Y and Z independently represent —O—, —S—, —C(—R)₂—, or —N(—R)—;
R, independently for each occurrence, represents H or lower alkyl;
R₁ represents one or more substituents, each independently selected from halogen, amino, acylamino, amidino, cyano, nitro, azido, ether, thioether, sulfoxido, -J-R₈, -J-OH, -J-lower alkyl, -J-lower alkenyl, -J-SH, -J-NH₂, or substituted or unsubstituted lower alkyl, lower alkenyl, cycloalkyl, heterocyclyl, cycloalkylalkyl, heterocyclylalkyl, aryl, heteroaryl, aralkyl, or heteroaralkyl, or protected forms of the above;
R8, independently for each occurrence, represents H or substituted or unsubstituted lower alkyl, cycloalkyl, heterocyclyl, aralkyl, heteroaralkyl, aryl, or heteroaryl;
J represents, independently for each occurrence, a chain having 0-8 units selected from —C(—R)₂—, —N(—R)—, —O—, and —S—;
m is an integer from 0 to 2;
n is an integer from 0 to 2;
p is 0 or 1;
q is an integer from 0 to 2; and
-Z-J-Hc, taken together, represent a substituted or unsubstituted nitrogen-containing heterocyclic or heteroaryl ring,
wherein said DAT-5HT2 antagonist has dopamine transport (DAT) inhibitory activity as well as 5HT₂ₐ receptor antagonist activity and/or 5HT_{2c} receptor antagonist activity; and
wherein said DAT-5HT2 antagonist is optionally provided as a packaged pharmaceutical comprising the DAT-5HT2 antagonist in an amount sufficient to treat an anxiety, depression or psychotic disorder and formulated in a pharmaceutically acceptable carrier with instructions (written and/or pictorial) describing the use of the formulation for treating a patient; and
wherein said packaged pharmaceutical is optionally characterized by one or more of the following:
the DAT-5HT2 antagonist is provided in a once-a-day formulation;
the packaged pharmaceutical is formulated for oral administration;
the DAT-5HT2 antagonist is formulated as a transdermal patch; or
the DAT-5HT2 antagonist is provided in an escalating dose which produces an escalating serum concentration of said DAT-5HT2 antagonist(s) over a period of at least 4 hours; or
wherein said DAT-5HT2 antagonist is optionally provided as a packaged pharmaceutical comprising the DAT-5HT2 antagonist in an amount sufficient to treat attention deficit disorder or attention-deficit hyperactivity disorder and formulated in a pharmaceutically acceptable carrier, with instructions (written and/or pictorial) describing the use of the formulation for treating a patient; or
wherein said DAT-5HT2 antagonist is optionally provided as a packaged pharmaceutical comprising:
(i) a mood-stabilizing formulation of the DAT-5HT2 antagonist,
(ii) a second drug selected from the group consisting of a serotonin reuptake inhibitor, a 5HT₆ receptor antagonist, an anticonvulsant, a norepinephrine reuptake inhibitor, an α-adrenoreceptor antagonist, an NK-3 antagonist, an NK-1 receptor antagonist, a PDE4 inhibitor, an Neuropeptide Y5 Receptor Antagonists, a D4 receptor antagonist, a 5HT_{1A} receptor antagonist, a 5HT_{1D} receptor antagonist, a CRF antagonist, a monoamine oxidase inhibitor, and a sedative-hypnotic drug, and
(iii) a label indicating the use of the packaged pharmaceutical for use in the treatment of a patient suffering from an anxiety, depression or psychotic disorder, and
wherein the DAT-5HT2 antagonist formulation and the second drug are optionally comingled in single dosage form.

2. The DAT-5HT2 antagonist of claim 1 characterized by one or more of the following:
Hc is a substituted or unsubstituted five-membered ring;
Hc represents a substituted or unsubstituted pyrrole, imidazole, triazole or pyridine;
Ar represents a bicyclic ring system in which at least one ring is aromatic;
Hc and Ar, if substituted, are substituted with one or more moieties selected from halogen, cyano, alkyl, alkenyl, alkynyl, aryl, hydroxyl, alkoxy, silyloxy, amino, nitro, thiol, amino, imino, amido, phosphoryl, phosphonate, carboxyl, carboxamide, silyl, thioether, alkylsulfonyl, arylsulfonyl, sulfoxide, selenoether, ketone, aldehyde, ester, or —(CH₂)ₘR₈, where m is an integer from 0 to 4;
J represents, independently for each occurrence, a chain having from 0-4 units selected from —C(—R)₂—, —N(—R)—, —O—, and —S—;
J represents, independently for each occurrence, a substituted or unsubstituted methylene or ethylene;
Y adjacent to Ar represents —O— or —S—, and preferably —O—;
Z represents —N(—R)—;
Z represents —N(H)— or —N(—CH₂)—;
Z, taken together with J and Hc, represents a heterocyclic ring attached to the core via a nitrogen atom; or
the heterocyclic ring is a substituted or unsubstituted piperidine, piperazine, or pyrrolidine ring.

3-19. (canceled)

20. Use in the manufacture of a pharmaceutical composition for prophylaxis or treatment of a patient susceptible to or suffering from a movement disorder, attention deficit disorder or attention-deficit hyperactivity disorder, a DAT-5HT2 antagonist represented by Formula I, or a pharmaceutically acceptable salt, solvate, metabolite or pro-drug thereof: 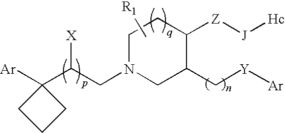
wherein, as valence and stability permit,
Ar, independently for each occurrence, represents a substituted or unsubstituted aryl or heteroaryl ring;
Hc represents a substituted or unsubstituted nitrogen-containing heteroaryl ring;
X represents H or OR;
Y and Z independently represent —O—, —S—, —C(—R)₂—, or —N(—R)—;
R, independently for each occurrence, represents H or lower alkyl;
R₁ represents one or more substituents, each independently selected from halogen, amino, acylamino, amidino, cyano, nitro, azido, ether, thioether, sulfoxido, -J-R₈, -J-OH, -J-lower alkyl, -J-lower alkenyl, -J-SH, -J-NH₂, or substituted or unsubstituted lower alkyl, lower alkenyl, cycloalkyl, heterocyclyl, cycloalkylalkyl, heterocyclylalkyl, aryl, heteroaryl, aralkyl, or heteroaralkyl, or protected forms of the above;
R8, independently for each occurrence, represents H or substituted or unsubstituted lower alkyl, cycloalkyl, heterocyclyl, aralkyl, heteroaralkyl, aryl, or heteroaryl;
J represents, independently for each occurrence, a chain having 0-8 units selected from —C(—R)₂—, —N(—R)—, —O—, and —S—;
m is an integer from 0 to 2;
n is an integer from 0 to 2;
p is 0 or 1;
q is an integer from 0 to 2; and
-Z-J-Hc, taken together, represent a substituted or unsubstituted nitrogen-containing heterocyclic or heteroaryl ring,
wherein said DAT-5HT2 antagonist has dopamine transport (DAT) inhibitory activity as well as 5HT₂ₐ receptor antagonist activity and/or 5HT_{2c} receptor antagonist activity; and
wherein said DAT-5HT2 antagonist is optionally characterized by one or more of the following:
Hc is a substituted or unsubstituted five-membered ring;
Hc represents a substituted or unsubstituted pyrrole, imidazole, triazole or pyridine;
Ar represents a bicyclic ring system in which at least one ring is aromatic;
Hc and Ar, if substituted, are substituted with one or more moieties selected from halogen, cyano, alkyl, alkenyl, alkynyl, aryl, hydroxyl, alkoxy, silyloxy, amino, nitro, thiol, amino, imino, amido, phosphoryl, phosphonate, carboxyl, carboxamide, silyl, thioether, alkylsulfonyl, arylsulfonyl, sulfoxide, selenoether, ketone, aldehyde, ester, or —(CH₂)ₘR₈, where m is an integer from 0 to 4;
J represents, independently for each occurrence, a chain having from 0-4 units selected from —C(—R)₂—, —N(—R)—, —O—, and —S—;
J represents, independently for each occurrence, a substituted or unsubstituted methylene or ethylene;
Y adjacent to Ar represents —O— or —S—, and preferably —O—;
Z represents —N(—R)—;
Z represents —N(H)— or —N(—CH₂)—;
Z, taken together with J and Hc, represents a heterocyclic ring attached to the core via a nitrogen atom; or
the heterocyclic ring is a substituted or unsubstituted piperidine, piperazine, or pyrrolidine ring.

21. A method for treating an anxiety, depression or psychotic disorder, attention deficit disorder, or attention-deficit hyperactivity disorder in a patient comprising administering to the patient a composition of a DAT-5HT2 antagonist represented by Formula I, or a pharmaceutically acceptable salt, solvate, metabolite or pro-drug thereof: 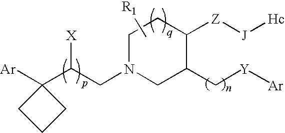
wherein, as valence and stability permit,
Ar, independently for each occurrence, represents a substituted or unsubstituted aryl or heteroaryl ring;
Hc represents a substituted or unsubstituted nitrogen-containing heteroaryl ring;
X represents H or OR;
Y and Z independently represent —O—, —S—, —C(—R)₂—, or —N(—R)—;
R, independently for each occurrence, represents H or lower alkyl;
R₁ represents one or more substituents, each independently selected from halogen, amino, acylamino, amidino, cyano, nitro, azido, ether, thioether, sulfoxido, -J-R₈, -J-OH, -J-lower alkyl, -J-lower alkenyl, -J-SH, -J-NH₂, or substituted or unsubstituted lower alkyl, lower alkenyl, cycloalkyl, heterocyclyl, cycloalkylalkyl, heterocyclylalkyl, aryl, heteroaryl, aralkyl, or heteroaralkyl, or protected forms of the above;
R8, independently for each occurrence, represents H or substituted or unsubstituted lower alkyl, cycloalkyl, heterocyclyl, aralkyl, heteroaralkyl, aryl, or heteroaryl;
J represents, independently for each occurrence, a chain having 0-8 units selected from —C(—R)₂—, —N(—R)—, —O—, and —S—;
m is an integer from 0 to 2;
n is an integer from 0 to 2;
p is 0 or 1;
q is an integer from 0 to 2; and
-Z-J-Hc, taken together, represent a substituted or unsubstituted nitrogen-containing heterocyclic or heteroaryl ring,
wherein said DAT-5HT2 antagonist has dopamine transport (DAT) inhibitory activity as well as 5HT₂ₐ receptor antagonist activity and/or 5HT_{2c} receptor antagonist activity; and
wherein said DAT-5HT2 antagonist is optionally characterized by one or more of the following:
Hc is a substituted or unsubstituted five-membered ring;
Hc represents a substituted or unsubstituted pyrrole, imidazole, triazole or pyridine;
Ar represents a bicyclic ring system in which at least one ring is aromatic;
Hc and Ar, if substituted, are substituted with one or more moieties selected from halogen, cyano, alkyl, alkenyl, alkynyl, aryl, hydroxyl, alkoxy, silyloxy, amino, nitro, thiol, amino, imino, amido, phosphoryl, phosphonate, carboxyl, carboxamide, silyl, thioether, alkylsulfonyl, arylsulfonyl, sulfoxide, selenoether, ketone, aldehyde, ester, or —(CH₂)ₘR₈, where m is an integer from 0 to 4;
J represents, independently for each occurrence, a chain having from 0-4 units selected from —C(—R)₂—, —N(—R)—, —O—, and —S—;
J represents, independently for each occurrence, a substituted or unsubstituted methylene or ethylene;
Y adjacent to Ar represents —O— or —S—, and preferably —O—;
Z represents —N(—R)—;
Z represents —N(H)— or —N(—CH₂)—;
Z, taken together with J and Hc, represents a heterocyclic ring attached to the core via a nitrogen atom; or
the heterocyclic ring is a substituted or unsubstituted piperidine, piperazine, or pyrrolidine ring.

22. The method of claim 21 characterized by one or more of the following:
the method is for the treatment of patients diagnosed with depression (e.g., episodic or recurrent major depressive disorders, dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, seasonal affective disorder, or bipolar disorders or manic depression);
the method is for the treatment of patients diagnosed with Bipolar Disorder, Bipolar Depression or Unipolar Depression;
the method is for the treatment of patients diagnosed with an anxiety disorder, e.g., an obsessive-compulsive disorder, a panic disorder, a psychoactive substance anxiety disorder, a post-traumatic stress disorder, a generalized anxiety disorder, a anxiety disorder NOS, an organic anxiety disorder, a phobia, or a substance-induced anxiety (e.g., induced by alcohol, amphetamines, caffeine, cannabis, cocaine, hallucinogens, inhalants, phencyclidine, sedatives, hypnotics, anxiolytics or other substance-induced, and adjustment disorders with anxiety or with mixed anxiety and depression); and
the method is for the treatment of patients diagnosed with a psychotic disorder (e.g., schizophrenia, schizophreniform diseases, acute mania, schizoaffective disorders, and depression with psychotic features).

23-34. (canceled)

35. A method for conducting a pharmaceutical business, comprising:
1) a. manufacturing the packaged pharmaceutical of claim 1; and
b. marketing to healthcare providers the benefits of using the package or preparation to treat patients suffering from an anxiety, depression or psychotic disorder, or from attention deficit disorder or attention-deficit hyperactivity disorder;
2) a. providing a distribution network for selling the packaged pharmaceutical of claim 1; and
b. providing instruction material to patients or physicians for using the package or preparation to treat patients suffering from an anxiety, depression or psychotic disorder, or from attention deficit disorder or attention-deficit hyperactivity disorder; or
3) a. determining an appropriate dosage of an DAT-5HT2 antagonist of claim 1 to enhance function performance in a class of patients suffering from an anxiety, depression or psychotic disorder, or from attention deficit disorder or attention-deficit hyperactivity disorder;
b. conducting therapeutic profiling of one or more formulations of the DAT-5HT2 antagonist identified in step (a), for efficacy and toxicity in animals; and
c. providing a distribution network for selling a the formulations identified in step (b) as having an acceptable therapeutic profile; and
optionally including an additional step of providing a sales group for marketing the preparation to healthcare providers.

36-38. (canceled)

39. A method for conducting a medical assistance reimbursement program, comprising:
a. providing a reimbursement program which permits, for prescription of a DAT-5HT2 antagonists of claim 1 for treating an anxiety, depression or psychotic disorder, or from attention deficit disorder or attention-deficit hyperactivity disorder, at least partial reimbursement to a healthcare provider or patient, or payment to a drug distributor;
b. processing one or more claims for prescription of an DAT-5HT2 antagonists for treating an anxiety, depression or psychotic disorder, or from attention deficit disorder or attention-deficit hyperactivity disorder; and
c. reimbursing the healthcare provider or patient, or paying a drug distributor, at least a portion of the cost of said prescription.
